(19) United States
(12) Patent Application Publication
(10) Pub. No.: US 2019/0209572 A1
(43) Pub. Date: Jul. 11, 2019

(54) BIFUNCTIONAL ANTIFUNGAL AGENTS AND METHODS OF TREATING FUNGAL INFECTION

(71) Applicants: YALE UNIVERSITY, NEW HAVEN, CT (US); MERCK SHARP & DOHME CORP, RAHWAY, NJ (US)

(72) Inventors: DAVID A. SPIEGEL, NEW HAVEN, CT (US); EGOR CHIRKIN, NEW HAVEN, CT (US); TERRY ROEMER, RAHWAY, NJ (US); PHILIPPE NANTERMET, RAHWAY, NJ (US)

(21) Appl. No.: 16/325,634

(22) PCT Filed: Aug. 18, 2017
(86) PCT No.: PCT/US2017/047536

§ 371 Date: Feb. 14, 2019

Related U.S. Application Data
(60) Provisional application No. 62/376,956, filed on Aug. 19, 2016.
(60) Provisional application No. 62/397,633, filed on Sep. 21, 2016.

(51) Int. Cl. A61K 31/53; C07D 251/68; C07D 403/12; A61K 31/10; A61P 31/10

(52) U.S. Cl. CPC A61K 31/53; C07D 251/68; A61P 31/10; A61K 31/10; C07D 403/12

(57) ABSTRACT

The present invention is directed to bifunctional compounds which are useful in the treatment of fungal infections. The present compounds contains at least one fungal binding moiety (FBM) which is linked to at least one antibody binding moiety (A_{B}M or ABT group) through a linker group, which optionally comprises a connector group. Compounds according to the present invention are useful in the treatment of fungal infections as described herein.

### RELATED APPLICATIONS

This application claims the benefit of priority of U.S. provisional application Ser. No. 62/376,956, filed 19 Aug. 2016 and U.S. 62/397,633, filed 21 Sep. 2016 of identical title, each of which applications is incorporated by reference in its entirety herein.

### FIELD OF THE INVENTION

The present invention is directed to Afunctional compounds which are useful in the treatment of fungal infections. The present compounds contains at least one fungal binding moiety (FBM) which is linked to at least one antibody binding moiety through a linker group, which optionally comprises a connector group. Compounds according to the present invention are useful in the treatment of fungal infections as described herein.

### BACKGROUND AND OVERVIEW OF THE INVENTION

Fungal infections represent a global public health threat.^{[1]} Systemic invasive mycoses are associated with a high mortality rate in immunocompromised individuals; immunosuppression arising from HIV infection, organ transplant and cancer therapy has led to a threefold increase in the incidence of fungal sepsis over the past three decades.^{[2]} These factors, coupled with the rapid spread of resistance lo available antifungals, makes novel therapeutic strategies to fight fungi highly desirable.^{[3]}

Fungi are eukaryotic, and therefore difficult to discriminate from human cells.^{[4]} The fungal cell wall, however, is absent from mammalian systems, so some of its component might constitute attractive targets for the development of selective therapies.^{[5]} Chitin, an amino-polysaccharide highly specific to the fungal cell wall is particularly compelling, and has already been employed for diagnosis of fungal infections^{[6]} and specific delivery of drugs into fungal cells.^{[7]}

In the present application, the inventors report the design, construction, and evaluation of Antibody-Recruiting Molecules (ARMs) to mediate killing of fungal cells by human effectors. As shown in FIG. 1, these ARMs are bifunctional small molecules consisting of two primary domains. The target-binding terminus (TBT) interacts with chitin, a fungal cell wall component consisting of repeating b-1,4-linked N-acetylglucosamine units.^{[8]} The antibody-binding terminus (ABT) recognizes and recruits anti-DNP antibodies, which are already present in the bloodstream in humans.^{[9]} Upon binding the pathogenic target cell, the bifunctional species recruits endogenous antibodies in the bloodstream, resulting in redirection of immune cells toward the target which had until then evaded immune surveillance. Indeed, the inventors have previously reported similar strategies for immunotherapy of cancer and HIV.^{[10]} Compared to antibodies, however, small-molecule immunotherapeutics are expected to exhibit low immunogenicity, high thermal stability, excellent penetration properties, and have the potential to be orally bioavailable.

### SUMMARY OF THE INVENTION

The present invention relates to anti-fungal compounds according to the general structure:

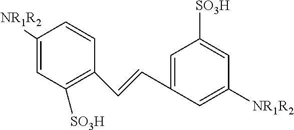

Wherein each R₁ is independently H, C₁-C₃ alkyl or a

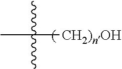

group (preferably H);

### Each R₂ is H, a

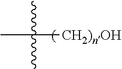

group or a

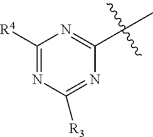

group, with the proviso that at

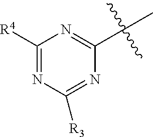

least one R₂ group is a group (preferably both R₂ groups
are

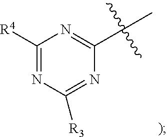

Each n′ is independently 1-6, preferably 1-3, more often 1 or 2;

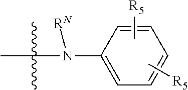

Each R₃ is independently H, Cl or a group (preferably when R₅ is CO₂R^{E} or SO₃H, each R₅ group may be independently ortho or meta substituted, or one R₅ is para substituted), preferably both R₃ groups are

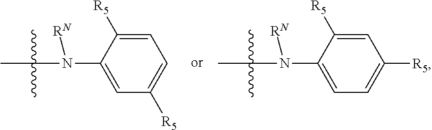

Where R^{N} is H or a C₁-C₃ alkyl group optionally substituted with one or two hydroxyl groups;
Each R₅ is independently H, CO₂R^{E}, SO₃H, L-P_{G} or L-ABT (preferably H, CO₂R^{E} or L-ABT);
R^{E} is H, a C₁-C₆ alkyl group or a L-ABT group;
P_{G} is a protecting group (preferably an amine protecting group, most preferably a BOC group);

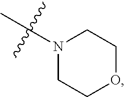 [SMILES: *N1CCOCC1]

Each R⁴ is independently H, Cl, a L-ABT group; or a

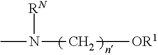

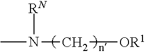

group, preferably at least one R⁴ is a group;
Where R^{N} and n′ are the same as above (preferably R^{N} is a C₁-C₃ alkyl group optionally substituted with one or two hydroxyl groups, preferably one hydroxyl group); and
R¹ is H, Pₒ (protecting group) or a L-ABT group;
Where L is a linker group optionally containing at least one connector group CT; and
ABT is an antibody binding moiety comprising a hapten which is capable of binding to an antibody present in a patient, preferably an antibody present in a patient prior to the administration of the compound to the patient, or
a pharmaceutically acceptable salt, stereoisomer, enantiomer, solvate or polymorph thereof.

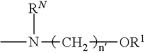

In certain preferred embodiments, R⁴ is L-ABT or where L is a group containing from 1-15 (preferably 1-10) PEG units or two separate PEG containing linker groups (from 1-15, preferably 1-10 PEG units) separated by a diamide group containing from 1 to 10 methylene groups separating the two amide groups; R^{N} is a C₁-C₃ alkyl group substituted with one or two hydroxyl groups), n′ is 1-3, more preferably 1 or 2 and R¹ is H, PG or a L-ABT group.

In certain embodiments, L is a polyethylene glycol linker optionally containing at least one CON group, comprising 1 to 100 ethylene glycol units, often 1-30 ethylene glycol units, often 1-20 ethylene glycol units, even more often 1-15 ethylene glycol units, 1-12 ethylene glycol units or 1, 2, 3, 4, 5, 6 7, 8, 9 or 10 ethylene glycol units.

In embodiments, L is a bond, at least one linker (preferably a single linker) which comprises a first linker group L1 which optionally includes a connector group CT and an optional linker group L2 which itself optionally includes a connector group CT, said first linker group L1 being linked to said second linker group L2 optionally (preferably) through a CT group; and ABT is preferably at least one dinitrophenyl group or is or contains a rhamnose group (preferably between 1 and 4 such groups).

In embodiments, L-ABT or LP_{G} is a group according to the chemical structure:

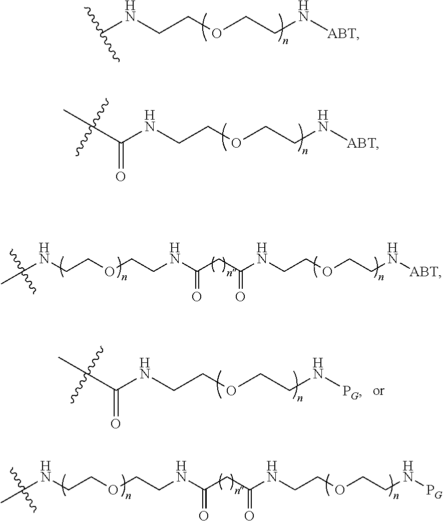

Where n is 1-45, often 1-30, 2-22, 2-14, 2-6, more often 2, 6, 14 and 22;
n^{41 is} 1-10, preferably 1-7, more often 2-6, often 3 or 6;
P_{G} is a protecting group, preferably a BOC group;
ABT is a DNP group or a rhamnose group, often a DNP group; or
a pharmaceutically acceptable salt, stereoisomer (a diastereomer or enantiomer), solvate or polymorph thereof.

Preferred compounds according to the present invention are represented by the chemical structure:

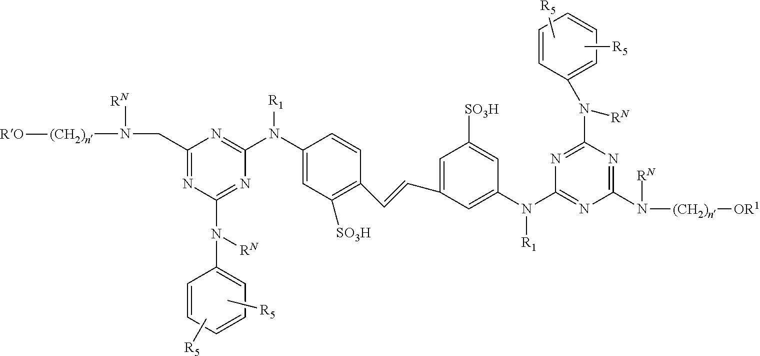

Where each R₁ and R^{N} is independently H or a C₁-C₃ alkyl group which is optionally substituted with one or two hydroxyl groups (preferably R¹ is H and R^{N} is a C₁-C₃ alkyl group which is optionally substituted with one or two hydroxyl groups);

R₅ is independently H, L-P_{G}, L-ABT (as depicted below) or SO₃H (if SO₃H, R₅ is disposed ortho, meta or para, preferably one R₅ is ortho and one meta at positions 2 and 5 of the phenyl ring or one R₅ is ortho and one para at positions 2 and 4 of the phenyl ring);
Each n′ is independently 1-6, preferably 1-3, more often 1 or 2; and
Each R¹ is H, L-P_{G} or L-ABT group as otherwise described herein, preferably a

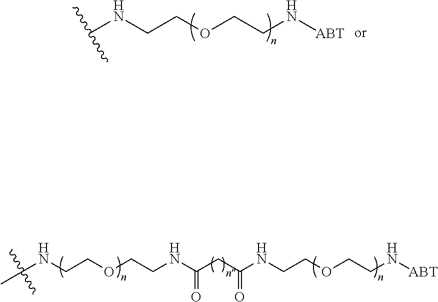

group;
n is 1-45, often 1-30, 2-22, 2-14, 2-6, more often 2, 6, 14 and 22;
n″ is 1-10, preferably 1-7, more often 2-6, often 3 or 6;
P_{G} is a protecting group;
ABT is an antibody binding group, preferably a DNP group or a rhamnose group, often a DNP group; or
a pharmaceutically acceptable salt, stereoisomer (a diastereomer or enantiomer), solvate or polymorph thereof.

In additional embodiments of the invention, a pharmaceutical composition comprises an effective amount of a compound as described above, optionally and preferably in combination with a pharmaceutically acceptable carrier, additive or excipient. In alternative aspects, pharmaceutical combination compositions comprise an effective amount of a compound as described herein, in combination with at least one additional agent which is used to treat a fungal infection or a secondary condition or effect of a fungal infection. These compounds, in combination, will often act synergistically in treating the fungal infection.

In a further aspect of the invention, compounds according to the present invention are used to treat a fungal infection in a patient. The method of treating a fungal infection comprises administering to a patient in need an effective amount of a compound as otherwise described herein in combination with a pharmaceutically acceptable carrier, additive or excipient, optionally in further combination with at least one additional anti-fungal agent which is effective in treating fungal infections, including drug resistant fungal infections, or one or more of its secondary conditions or effects. The method of treatment may be combined with alternative anti-fungal treatments, such as the use of traditional anti-fungal agents, and non-traditional anti-fungal agents such as oil of oregano, tea tree oil, caprylic acid, tumeric/curcumin, and the like.

The present invention also relates to a method for treating a fungal infection or inhibiting the effects of a fungal infection, to reduce the likelihood or inhibit the spread of the fungal infection into other tissues of the patients' body.

Pursuant to the present invention, synthetic compounds for controlling or creating human immunity have the potential to revolutionize anti-fungal treatment. Motivated by challenges in this arena, the present inventors provide a strategy to target fungi for immune-mediated destruction by targeting the fungi with an agent which selectively binds to polysaccharides and proteins present in fungus (e.g. chitin, β-mannosides, α-mannosides, β-glucans and proteins) and contains a moiety which attracts and binds to endogenous antibodies already present in a patient or subject to be treated. This bifunctional construct is formed by selectively, covalently attaching an antibody-binding small molecule to the polysaccharides and proteins present in the fungus. The present inventors demonstrate that ARM-F compounds are capable of redirecting antibodies to the surfaces of target fungal cells infecting a patient or subject and mediating an antibody response in combination with specific antifungal activity associated with the binding of the fungal cells by the compounds. The present invention represents a novel technology has significant potential to impact the treatment of a variety of harmful fungal infections.

### BRIEF DESCRIPTION OF THE FIGURES

FIG. 1 shows the design of antifungal antibody-recruiting small molecules (ARM-F targeting chitin.

FIG. 2, Scheme 1, summarizes a synthetic approach to a number of compounds related to the present invention. Reagents and conditions which are employed for the several steps: (a) cyanuric chloride, H₂O/acetone or H₂O/methyl ethyl ketone, pH 4.5-5.5, 0° C. to +5° C.;

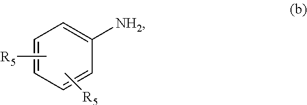

H₂O/acetone or H₂O/methyl ethyl ketone, pH 6-7, 40-45° C.;

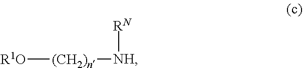

H₂O/acetone or H₂O/methyl ethyl ketone, pH 6-7, 85-95° C., then 1M aq HCl, 0° C. (yield: 5-15% over 3 steps).

FIG. 3, Scheme 2, summarizes a synthetic approach to a number of alternative compounds which are related to the present invention. Reagents and conditions: (a) cyanuric chloride, H₂O/acetone or H₂O/methyl ethyl ketone, pH 4.5-5.5, 0° C. to +5° C.;

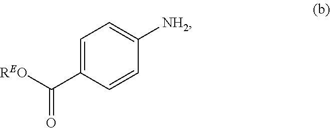

H₂O/acetone or H₂O/methyl ethyl ketone, pH 6-7, 40-45° C.;

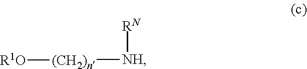

H₂O/acetone or H₂O/methyl ethyl ketone, pH 6-7, 85-95° C., then 1M aq HCl, 0° C. (yield: 1-15% over 3 steps).

FIG. 4, Scheme 3 summarizes a synthetic approach to a number of alternative compounds which are related to the present invention. Reagents and conditions: (a) cyanotic chloride, H₂O/acetone or H₂O/methyl ethyl ketone, pH 4.5-5.5, 0° C. to +5° C.;

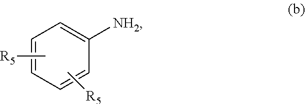

H₂O/acetone or H₂O/methyl ethyl ketone, pH 6-7, 40-45° C.; (c) diethanolamine, H₂O/acetone or H₂O/methyl ethyl ketone, pH 6-7, 85-95° C.; (d) Zn, acetic acid, 0° C. to 80° C.; (e) cyanuric chloride, H₂O/acetone or H₂O/methyl ethyl ketone, pH 4.5-5.5, 0° C. to +5° C.;

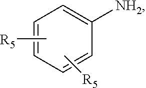

H₂O/acetone or H₂O/methyl ethyl ketone, pH 6-7, 40-45° C.;

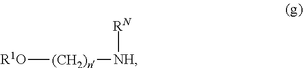

H₂O/acetone or H₂O/methyl ethyl ketone, pH 6-7, 85-95° C., then 1M aq HCl, 0° C. (yield: 1-3% over 7 steps).

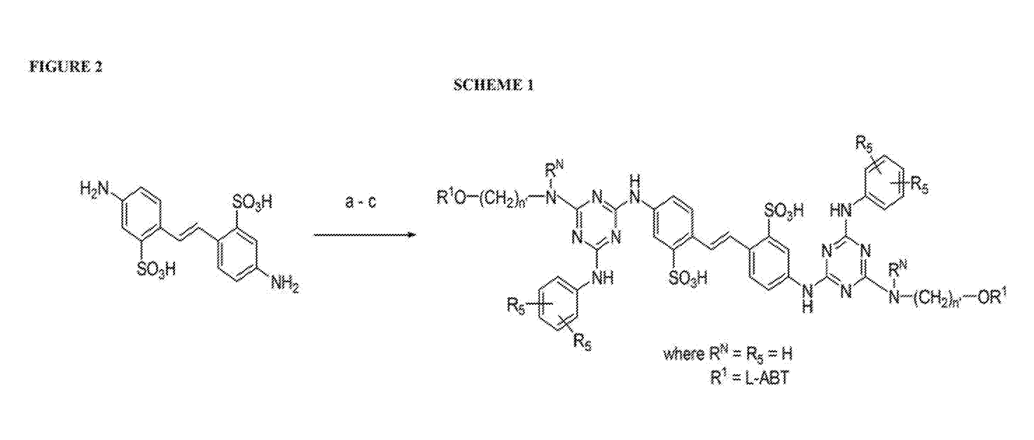

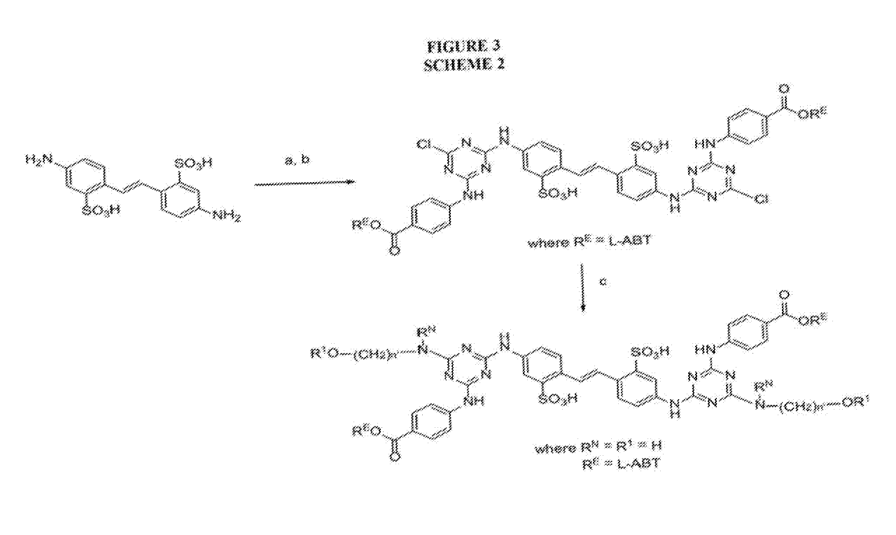

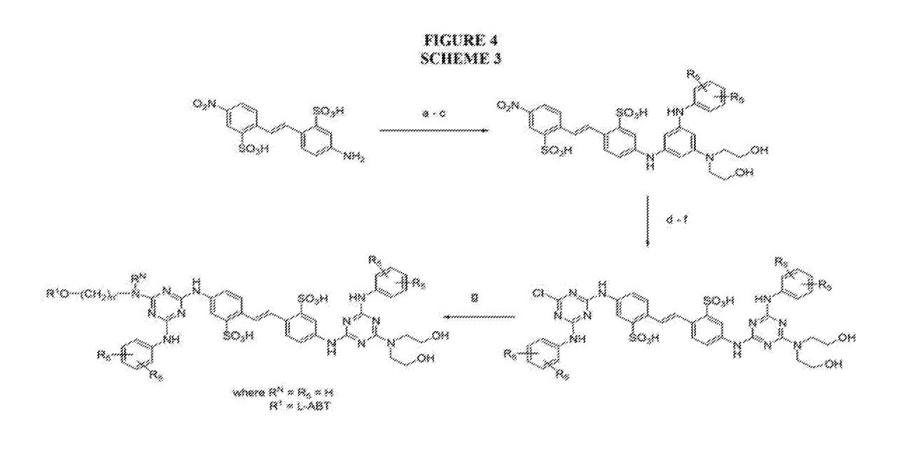

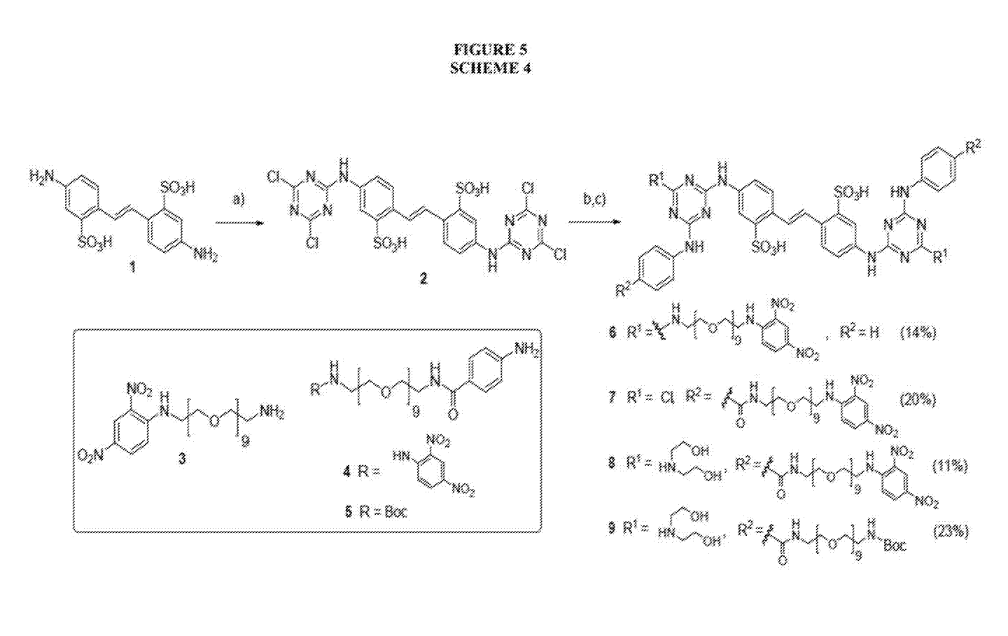

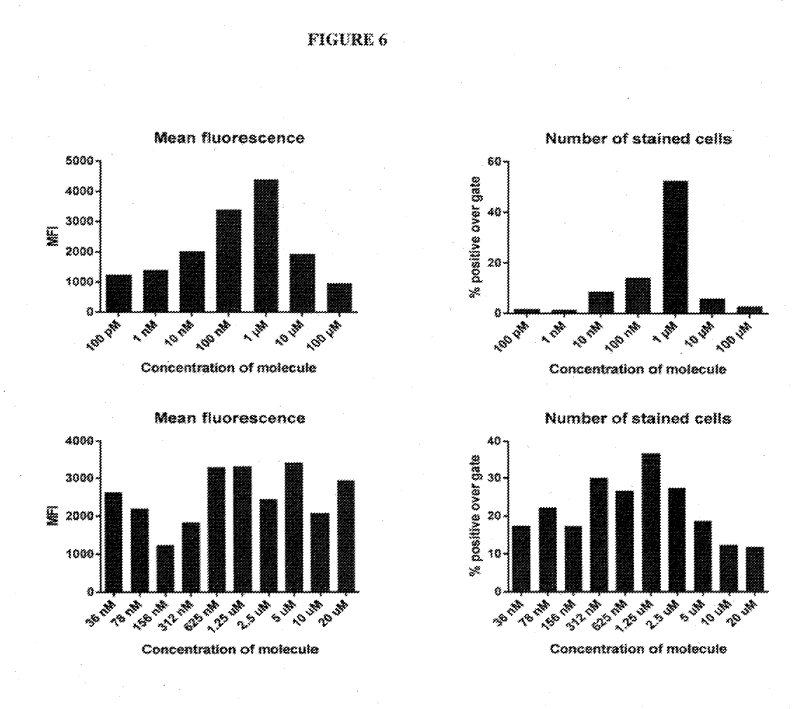

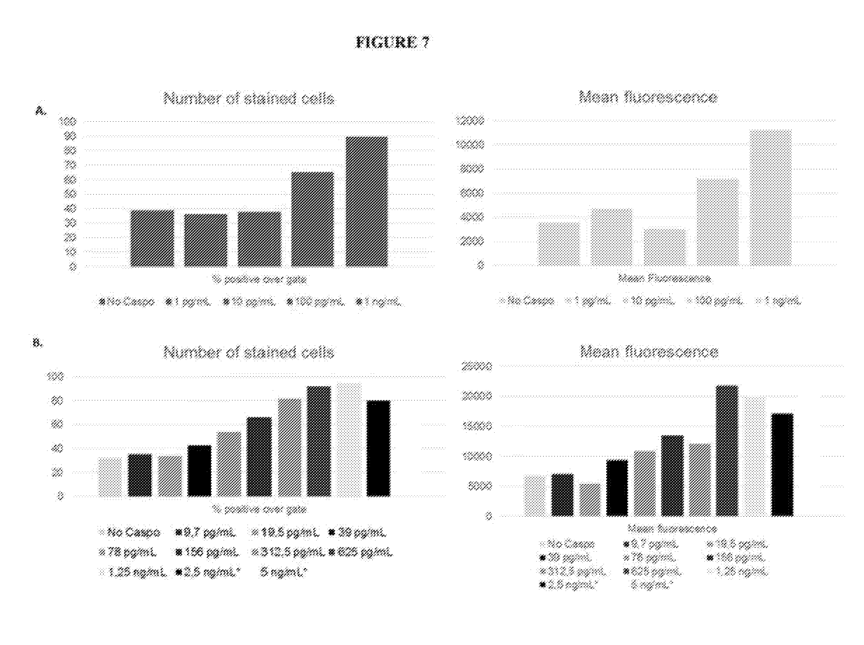

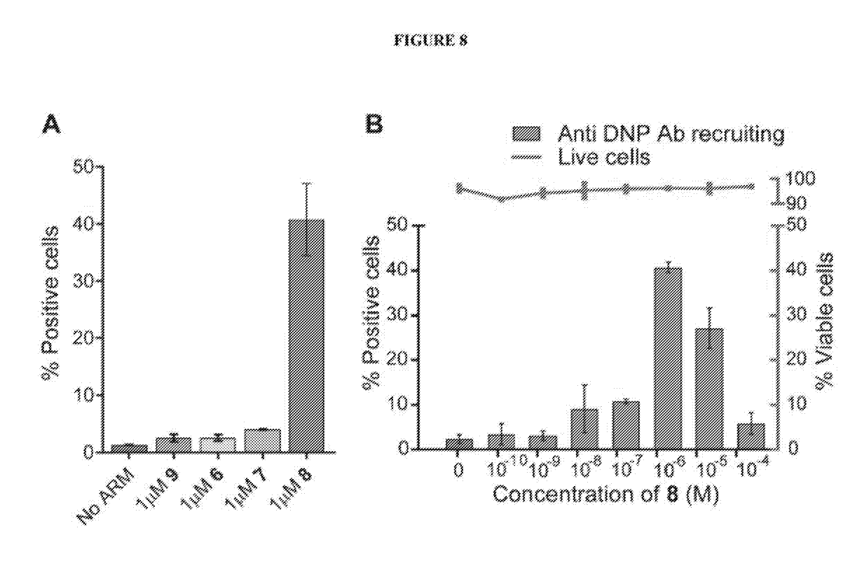

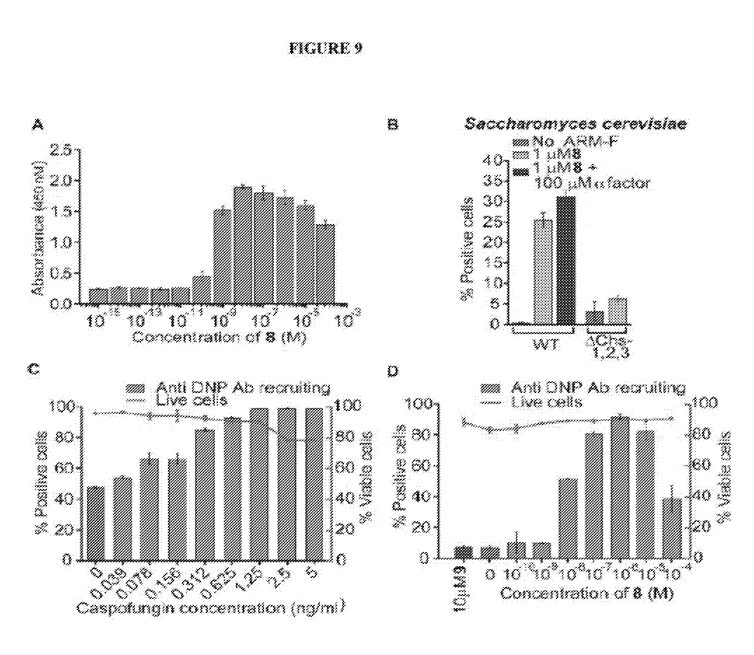

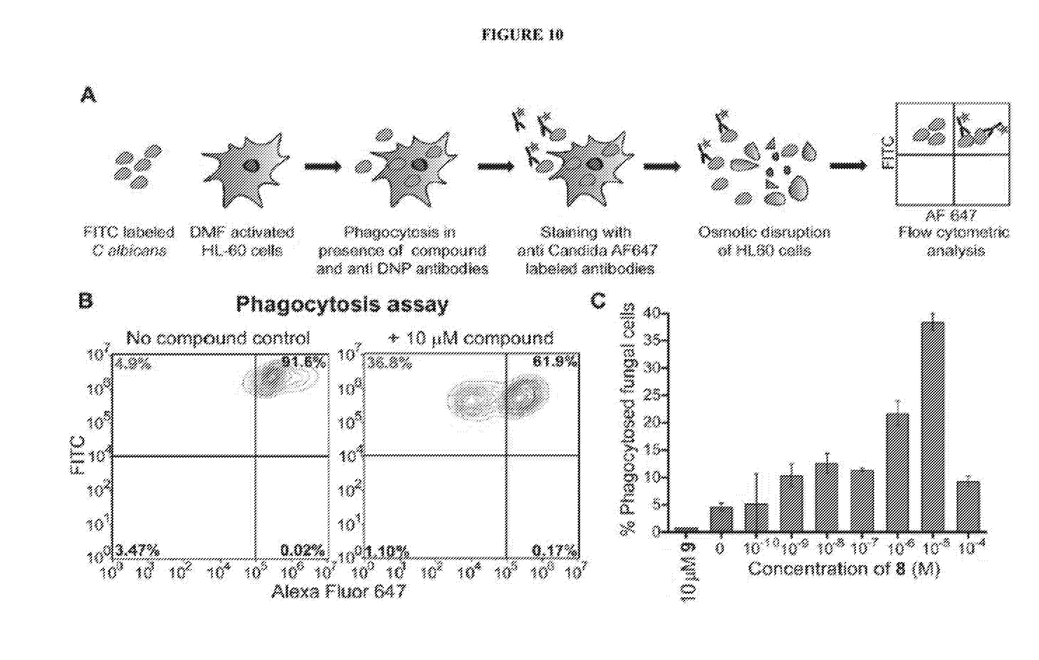

FIG. 5, Scheme 4 summarizes the compounds presented in FIGS. 2-4 in a summary scheme. Reagents and conditions: a) cyanuric chloride, H₂O, MEK, 0° C., pH 4-5; b) 6: aniline, H₂O, MEK, 45° C., pH 6-7; 7-8: 4, H₂O, MEK, 45° C., pH 6-7; 9:5, H₂O, MEK, 45° C., pH 6-7; c) 6:3, H₂O, MEK, 85° C., pH 8-9; 7-9: DEA, H₂O, MEK, 85° C., pH 8-9. Boc=tert-butyloxycarbonyl, DEA=diethanolamine, MEK=methyl ethyl ketone.

FIG. 6 shows the antibody-recruiting activity of the prepared derivatives evaluated on Candida albicans using flow cytometry. The compound F8 was found to recruit the anti-DNP antibodies in a dose-dependent manner, with the highest activity reached at 1 μM concentration.

FIG. 7 shows the evaluation of compound F8 to recruit the anti-DNP antibodies in the presence of Caspofungin (Cancidas®). The carbohydride polymers glucan and chitin are two interrelated elements of fungal cell wall which are critical to maintaining its physical integrity. The inventors envisaged that the combination of ARM-F agent with the (1→3)-β-D-glucan synthesis inhibitor Caspofungin would result in a synergistic effect through (i) the morphological changes of fungal cell wall leading to higher surface exposure of chitin and (ii) enhanced chitin synthesis. Indeed, the antibody-recruitment was enhanced in the presence of Caspofungin, with the maximum observed at 1 ng/mL concentration of Caspofungin.

FIG. 8 shows antibody recruitment to fungal cell surface by flow cytometry A. Screening of potential ARM-F candidates. B. Dose-dependent recruitment of anti-DNP antibodies by ARM-F (8) with no loss of viability.

FIG. 9 shows that the binding of ARM-F (8) is dependent on cell wall chitin. A. shows binding of 8 to immobilized chitin in ELISA. B. Shows chitin dependent activity of 8 in a S. cerevisiae chitin synthase knock-out model. C. shows the effect of caspofungin treatment on antibody recruitment by 8. D. Dose-dependent antibody recruitment to C. albicans pretreated with 1.25 nM caspofungin.

FIG. 10 shows antibody-dependent cellular phagocytosis assays. A. shows a schematic depiction, of flow cytometry-based functional phagocytosis assay. B. shows representative cytometry plots illustrating the distribution of C. albicans cell populations post phagocytosis in the presence/absence of 8. C. shows ARM-F (8) induces a dose-dependent opsono-phagocytosis of C. albicans.

### DETAILED DESCRIPTION OF THE INVENTION

In accordance with the present invention there may be employed conventional chemical synthetic and pharmaceutical formulation methods, as well as pharmacology, molecular biology, microbiology, and recombinant DNA techniques within the skill of the art. Such techniques are well-known and are otherwise explained fully in the literature.

Where a range of values is provided, it is understood that each intervening value, to the tenth of the unit of the lower limit unless the context clearly dictates otherwise (such as in the case of a group containing a number of carbon atoms), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It is to be noted that as used herein and in the appended claims, the singular forms “a,” “an”, “and” and “the” include plural references unless the contest clearly dictates otherwise.

Furthermore, the following terms shall have the definitions set out below. It is understood that in the event a specific term is not defined hereinbelow, that term shall have a meaning within its typical use within context by those of ordinary skill in the art.

The term “compound”, as used herein, unless otherwise indicated, refers to any specific chemical compound disclosed herein and includes tautomers, regioisomers, geometric isomers, stereoisomers and where applicable, optical isomers (enantiomers) thereof, as well as pharmaceutically acceptable salts and derivatives (including prodrug forms) thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, within context, to prodrug forms of compounds which have been modified to facilitate the administration and delivery of compounds to a site of activity. It is noted that in describing the present compounds, numerous substituents, linkers and connector molecules and variables associated with same, among others, are described. The use of a bond presented as—signifies that a single bond is present or absent, depending on the context of the chemistry described. The use of a bond presented as—signifies that a single bond or a double bond is intended depending on the context of the chemistry described. It is understood by those of ordinary skill that molecules which are described herein are stable compounds as generally described hereunder. Active compounds according to the present invention which bind to fungi and attract antibodies are collectively referred to as ARM-F compounds, as well as difunctional compounds (even where the compounds are multifunctional).

The term “patient” or “subject” is used throughout the specification within context to describe an animal, generally a mammal and preferably a human, to whom treatment, including prophylactic treatment (prophylaxis, including especially as that term is used with respect to reducing the likelihood of the spread of a fungal infection), with the compositions according to the present invention is provided. For treatment of those infections, conditions or disease states which are specific for a specific animal such as a human patient or a patient of a particular gender, such as a human male or female patient, the term patient refers to that specific animal. Compounds according to the present invention are useful tor the treatment of fungal infections and conditions, including especially for use in reducing the likelihood of the spread of a fungal infection.

The term “effective” is used herein, unless otherwise indicated, to describe an amount of a compound or composition which, in context, is used to produce or effect an intended result, whether that result relates to the inhibition of the effects of a disease state (e.g. a fungal infection and/or condition) on a subject or the treatment or prophylaxis of a subject for secondary conditions, disease states or manifestations of disease states as otherwise described herein. This term subsumes all other effective amount or effective concentration tarns (including the term “therapeutically effective”) which are otherwise described in the present application.

The terms “treat”, “treating”, and “treatment”, etc., as used herein, refer to any action providign a benefit to a patient at risk for a fungal infection, including improvement in the condition through lessening or suppression of at least one symptom, inhibition of fungal growth, reduction in fungal cells, prevention, reduction in the likelihood or delay in progression of the spread of a fungal infection, prevention or delay in the onset of disease states or conditions which occur secondary to fungal infections, among others. Treatment, as used herein, encompasses both prophylactic and therapeutic treatment, within context. The term “prophylactic” when used, means to reduce the likelihood of an occurrence or the severity of an occurrence (including the spread of a fungal infection) within the context of the treatment of a fungal infection, including a fungal infection as otherwise described hereinabove.

The term “fungal” or “fungus infection” is used to describe any of a diverse group of eukaryotic single-celled or multinucleate organisms or an infection caused thereby that live by decomposing and absorbing the organic material in which they grow. Fungi pursuant to the present invention comprise mushrooms, molds, mildews, smuts, rusts, and yeasts, for purposes of the present invention principally molds and yeasts and classified in the kingdom Fungi or, in some alternative classification systems, in the division Thallophyta of the kindom Plantae, which cause infections.

Fungal infections which may be treated by compounds and/or compositions according to the present invention include, for example, dermatological fungal diseases and/or conditions, respiratory fungal diseases and/or conditions, neurological fungal diseases and/or conditions and hepatic fungal diseases and/or conditions. Dermatologic fungal diseases and/or conditions are caused principally by a group of fungi commonly referred to as dermatophytes, including for example, Tinea versicolor (caused by P. orbiculare, or P. ovale), Athlete's Foot (Tinea pedis), Jock Itch (Tinea cruris), Ringworm of the body (Tinea corporis), Tinea of the beard (Tinea barbae) and Tinea of the scalp (Tinea capitis), among others. Respiratory fungal diseases and/or conditions and the fungus which is the infective agent for such diseases and/or conditions include Histoplasmosis (H. capsulatum), Blastomycosis (B. dermatitidis), Coccidiodomycosis (C. immitis, or Coccidiodes posadasii), Paracoccidiodomycosis (Paracoccidioides brasiliensis), Cryptococcosis (Cryptococcus neoformans, or Cryptococcus gattii), Aspergillosis (Aspergillus spp), Zygomycosis (caused by members of the genera Mucor, Rhizopus, or Absidia), Candidiasis (C. albicans, C. tropicalis, or C. glabrata), Pneumocystis pneumonia (Pneumocystis jirovecii). Fungal respiratory diseases and/or conditions range in severity from asymptomatic, to presentation with mild malaise, to life threatening respiratory disease. Neurological fungal diseases and/or conditions including for example meningitis (caused by Cryptococcus spp, Aspergillus spp., Pseudallescheria boydii, Coccidiodies spp., Blastomyces dermatitidis, and Histoplasma capsulatum) and Brain Abscess (caused by Candida spp., Aspergillus spp., Rhizopus spp., Mucor spp., P. boydii) often seen, in immunocompromised individuals. Hepatic fungal diseases and/or conditions and agents which cause such diseases and/or conditions include Histoplasmosis (Histoplasma capsulatum) and Candidiasis (Candida spp).

The term “additional anti-fungal agent” is used to describe a traditional or non-traditional anti-fungal agent which can be combined with compounds according to the present invention either in a single composition or as a co-administered combination in treating fungal infections pursuant to the present invention. Additional anti-fungal agents include, for example, the polyenes, imidazoles, triazoles, allylamines, and echinocandins, as well as miscellaneous anti-fungal agents. Polyene antifungals include nystatin and amphotericin B. imidazole antifungal drugs include ketoconazole and clotrimazole. Triazole antifungal agents include fluconazole, itraconazole, posaconazole and voriconazole. Allylamines include terbinafine. Echinocandins include anidulafungin, caspofungin and micafungin. Miscellaneous anti-fungal agents include flucytosine, griseofulvin and petamine. Non-traditional anti-fungal agents such as oil of oregano, tea tree oil, caprylic acid, tumeric/curcumin, and the like may also be included in compositions and methods according to the present invention.

The term “antibody binding moiety”, “antibody binding terminus” or “antibody binding structure” (A_{B}M or ABT, which abbreviations are used synonymously) within the general formula of compounds according to the present invention) is used to described that portion of a bifunctional ARM-F compound according to the present invention which comprises at least one small molecule or hapten which can bind to antibodies within the patient. The term “hapten” is used to describe a small-molecular-weight inorganic or organic molecule that alone is not antigenic but which when linked to another molecule, such as a carrier protein (albumin, etc.) or in the case of the present invention, as an antibody terminus in the present compounds, is antigenic; and an antibody raised against the hapten (generally, the hapten bonded or complexed to the carrier) will react with the hapten alone. Because, in many instances and preferably, anti-hapten (especially anti-DNP and anti-rhamnose) antibodies are already present in the human blood stream as endogenous antibodies because they naturally become raised to endogenous haptens (already present in patients), no pre-vaccination is necessary for ARM-F activity, but vaccination/raising immunogenicity in a patient may optionally be used to increase the efficacy of the ARM-F compounds disclosed herein.

It is preferred that the antibody binding moiety comprise a hapten which is reactive with (binds to) an endogenous antibody that pre-exists in the patient prior to initiation of therapy with the compounds of the present invention and does not have to be separately raised as part of a treatment regimen (for example, by vaccination or other approaches for enhancing immunogenicity), which is optionally used in the present invention. Thus, haptens which comprise a di-or trinitro phenyl group or a rhamnose group, or a digalactose hapten (Gal-Gal-Z, preferably Gal-Gal-sugar, preferably Gal-Gal-Glu), are preferred. Additionally, a compound according to the general structure:

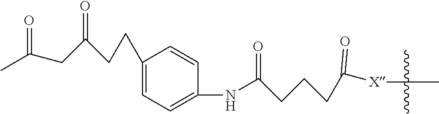

Where X″ is O, CH₂, NR¹, S; and
R¹ is H, a C₁-C₃ alkyl group or a —C(O)(C₁-C₃) group;
May be used as haptens in the present invention.

Further, a moiety according to the chemical structure:

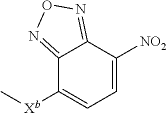

Where X^{b} is a bond, O, CH₂, NR¹ (as above) or S may also be used as a hapten (A_{B}M) in the present invention.

A preferred A_{B}M moiety is:

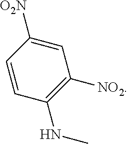

Additional A_{B}M moieties include the following:

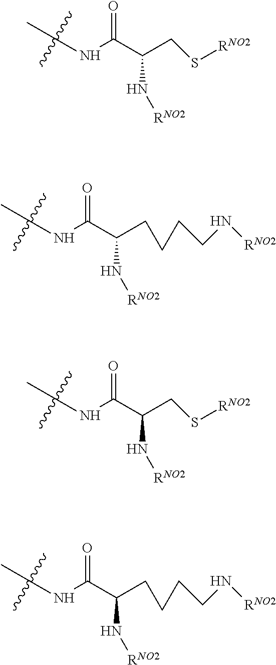

Where R^{N02} is a nitrophenyl group or a dinitrophenyl group which is bonded to the adjacent amine group or thio group as indicated;

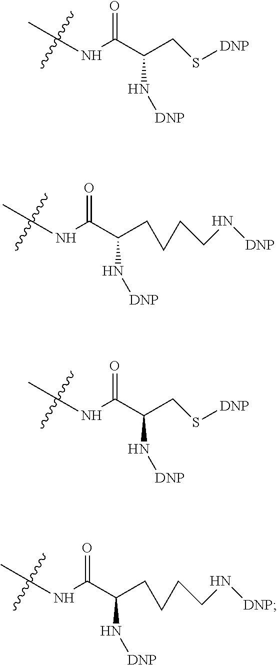

a group according to the chemical structure:

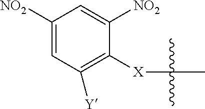

Where Y′ is H or NO₂ (preferably H);
X is O, CH₂, NR¹, S, S(O), S(O)₂, —S(O)₂O, —OS(O)₂, or OS(O)₂O; and
R¹ is H, a C₁-C₃ alkyl group, or a —C(O)(C₁-C₃) group;

The (Gal-Gal-Z) hapten is represented by the chemical formula:

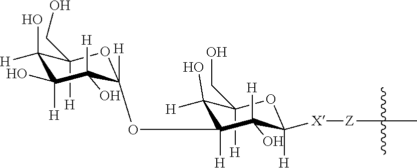

Where X′ is CH₂, O, N—R¹′, or S, preferably O;
R^{1′} is H or C₁-C₃ alkyl; and
Z is a bond, a monosaccharide, disaccharide, oligosaccharide, glycoprotein or glycolipid, preferably a sugar group, more preferably a sugar group selected from the monosaccharides, including aldoses and ketoses, and disaccharides, including those disaccharides described herein. Monosaccharide aldoses include monosaccharides such as aldotriose (D-glyceraldehdye, among others), aldotetroses (D-erythrose and D-Threose, among others), aldopentoses, (D-ribose, D-arabinose, D-xylose, D-lyxose, among others), aldohexoses (D-allose, D-altrose, D-Glucose, D-Mannose, D-glose, D-idose, D-galactose and D-Talose, among others), and the monosaccharide ketoses include monosaccharides such as ketotriose (dihydroxyacetone, among others), ketotetrose (D-erythrulose, among others), ketopehtose (D-ribulose and D-xylulose, among others), ketohexoses (D-Psicone, D-Fructose, D-Sorbose, D-Tagatose, among others), aminosugars, including galactoseamine, sialic acid, N-acetylglucosamine, among others and sulfosugars, including sulfoquinovose, among others. Exemplary disaccharides which find use in the present invention include sucrose (which may have the glucose optionally N-acetylated), lactose (which may have the galactose and/or the glucose optionally N-acetylated), maltose (which may have one or both of the glucose residues optionally N-acetylated), trehalose (which may have one or both of the glucose residues optionally N-acetylated), cellobiose (which may have one or both of the glucose residues optionally N-acetylated), kojibiose (which may have one or both of the glucose residues optionally N-acetylated), nigerose (which may have one or both of the glucose residues optionally N-acetylated), isomaltose (which may have one or both of the glucose residues optionally N-acetylated), β,β-trehalose (which may have one or both of the glucose residues optionally N-acetylated), sophorose (which may have one or both of the glucose residues optionally N-acetylated), Iaminaribiose (which may have one or both of the glucose residues optionally N-acetylated), gentiobiose (which may have one or both of the glucose residues optionally N-acetylated), turanose (which may have the glucose residue optionally N-acetylated), maltulose (which may have the glucose residue optionaily N-acetylated), palatinose (which may have the glucose residue optionally N-acetylated), gentiobiluose (which may have the glucose residue optionally N-acetylated), mannobiose, melibiose (which may have the glucose residue and/or the galactose residue optionally N-acetylated), melibiulose (which may have the galactose residue optionally N-acetylated), rutinose, (which may have the glucose residue optionally N-acetylated), rutinulose and xylobiose, among others. Oligosaccharides for use in the present invention as Z can include any sugar of three or more (up to about 100) individual sugar (saccharide) units as described above (i.e., any one or more saccharide units described above, in any order, especially including glucose and/or galactose units as set forth above), or for example, fructo-oligosaccharides, galactooligosaccharides and mannan-oligosaccharides ranging from three to about ten-fifteen sugar units in size. Glycoproteins for use in the present invention include, for example, N-glycosylated and O-glycosylated glycoproteins, including the mucins, collagens, transferrin, ceruloplasmin, major histocompatability complex proteins (MHC), enzymes, lectins and selectins, calnexin, calreticulin, and integrin glycoprotein IIb/IIa, among others. Glycolipids for use in the present invention include, for example, glyceroglycolipids (galactolipids, sulfolipids), glycosphingolipids, such as cerebrosides, galactocerebrosides, glucocerebrosides (including glucobicaranateoets), gangliosides, globosides, sulfatides, glycophosphphingolipids and glycocalyx, among others.

Preferably, Z is a bond (linking a Gal-Gal disaccharide to a linker or connector molecule) or a glucose or glucosamine (especially N-acetylglucosamine).

It is noted that Z is linked to a galactose residue through a hydroxyl group or an amine group on the galactose of Gal-Gal, preferably a hydroxyl group. A preferred hapten is Gal-Gal-Glu which is represented by the structure:

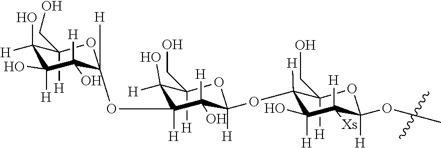

### Where Xs is OH or NHAc.

Other ABT groups include, for example, the following groups:

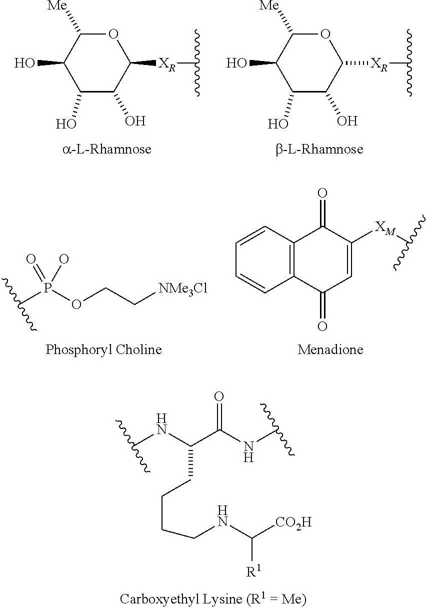

Where X_{R} is O, S or NR¹; and
X_{M} is O, NR¹ or S, and
R¹ is H, a C₁-C₃ alkyl group or a —C(O)(C₁-C₃) group,
or a pharmaceutically acceptable salt form or alternative salt form thereof.

Noted is that more than one dinitrophenyl group or rhamnose group (preferably from 1 to 4 of these groups) may be used in the present compounds to provide enhanced antibody recruitment activity.

It is noted in the carboxyethyl lysine A_{B}M moiety either one, two or three of the nitrogen groups may be linked to the remaining portion of the molecule through the linker or one or both of the remaining nitrogen groups may be substituted with a dinitrophenyl through an X group as otherwise described herein.

The term “pharmaceutically acceptable salt” is used throughout the specification to describe a salt form of one or more of the compounds herein which are presented to increase the solubility of the compound in saline for parenteral delivery or in the gastric juices of the patient's gastrointestinal tract in order to promote dissolution and the bioavailability of the compounds. Pharmaceutically acceptable salts including those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be particularly preferred as neutralization salts of carboxylic acid containing compositions according to the present invention. The term “salt” shall mean any salt consistent with the use of the compounds according to the present invention. In the case where the compounds are used in pharmaceutical indications, including the treatment of HIV infections, the term “salt” shall mean a pharmaceutically acceptable salt, consistent with the use of the compounds as pharmaceutical agents.

The term “linker”, “L”, “L1” or “L2” refers to a chemical entity connecting an antibody binding (A_{B}M) moiety to a fungal binding moiety FBM, optionally through at least one (preferably one) connector moiety (CT) through covalent bonds. The linker between the two active portions of the molecule, that is the antibody binding moiety (A_{B}M) and the fungal binding moiety FBM ranges from about 5 Å to about 50 Å or more in length, about 6 Å to about 45 Å in length, about 7 Å to about 40 Å in length, about 8 Å to about 35 Å in length, about 9 Å to about 30 Å in length, about 10 Å to about 25 Å in length, about 7 Å to about 20 Å in length, about 5 Å to about 16 Å in length, about 5 Å to about 15 Å in length, about 6 Å to about 14 Å in length, about 10 Å to about 20 Å in length, about 11 Å to about 25 Å in length, etc. linkers which are based upon ethylene glycol units and are between 2 and 15 glycol units, 1 and 8 glycol units, 1, 2, 3, 4, 5, and 6 glycol units in length may be preferred. By having a linker with a length as otherwise disclosed herein, the A_{B}M moiety may be situated to advantageously take advantage of the biological activity of compounds according to the present invention which bind to fungal cells and attract endogenous antibodies to those fungal cells to which the compounds are bound, resulting in the selective and targeted death of those cells. The selection of a linker component is based on its documented properties of biocompatibility, solubility in aqueous and organic media, and low immunogenicity/antigenicity. Although numerous linkers may be used as otherwise described herein, a linker based upon polyethyleneglycol (PEG) linkages, polypropylene glycol linkages, or polyethyleneglycol-co-polypropylene oligomers (up to about 100 units, about 1 to 100, about 1 to 75, about 1 to 60, about 1 to 50, about 1 to 35, about 1 to 25, about 1 to 20, about 1 to 15, 2 to 10, about 4 to 12, about 1 to 8, 1 to 3, 1 to 4, 2 to 6, 1 to 5, etc.) may be favored as a linker because of the chemical and biological characteristics of these molecules. The use of polyethylene (PEG) linkages is preferred. When describing linkers according to the present invention, including polyethylene glycol linkers or other linkers, one or more additional groups (e.g., methylene, groups, amide groups, amine groups, etc. where the amine is substituted with H or a C₁-C₃ alkyl group etc., amide or amine groups are often preferred) may be covalently attached at either end of the linker group to attach to a FBM group, a CT group, another linker group or an A_{B}M group.

Alternative linkers may include, for example, polyamino acid linkers of up to 100 amino acids (of any type, preferably D- or L-amino acids, preferably naturally occurring L-amino acids) in length (m is about 1 to 100, about 1 to 75, about 1 to 60, about 1 to 50, about 1 to 45, about 1 to 35, about 1 to 25, about 1 to 20, about 1 to 15, 2 to 10, about 4 to 12, about 5 to 10, about 4 to 6, about 1 to 8, about 1 to 6 , about 1 to 5, about 1 to 4, about 1 to 3, etc.), optionally including one or two connecting groups (preferably at one or both ends of the polyamino acid linker).

Alternative linkers include those according to the following chemical structure:

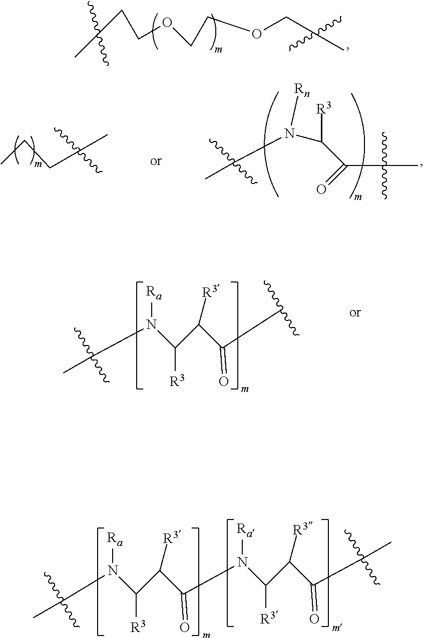

Where Rₐ and R_{a′} are each independently H, C₁-C₃ alkyl, alkanol, aryl or benzyl or form a cyclic ring with R³ or R^{3′} on a carbon adjacent to the nitrogen (to form a pyrrolidine-proline or hydroxpyrrolidine-hydroxyproline group) and R³, R^{3′} and R^{3″} are each independently a side chain derived from an amino acid preferably selected from the group consisting of alanine (methyl), arginine (propyleneguanidine), asparagine (methylenecarboxyamide), aspartic acid (ethanotic acid), cysteine (thiol, reduced or oxidized di-thiol), glutamine (ethylcarboxyamide), glutaimc acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline or hydroxyproline (R³ or R^{3′} forms a cyclic ring with Rₐ or R_{a′} respectively and the adjacent nitrogen group to form a pyrrolidine group-proline or a hydroxypyrrolidine group-hydroxyproline), serine (methanol), threonine (ethanol, 1-hydroxyethane), tryptophan (methyleneindole), tyrosine (methylene phenol) or valine (isopropyl) and m and m′ (within the context of this use) is each independently an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5.

Preferred linkers include those according to the chemical structures:

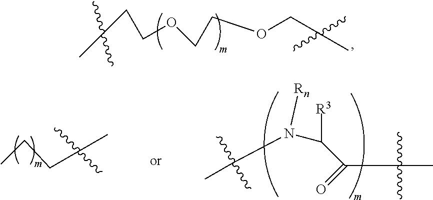

Where each m is independently 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5, or is a polypropylene glycol or polypropylene-co-polyethylene glycol linker having between 1 and 100 alkylene glycol units;

Another linker according to the present invention comprises a polyethylene glycol linker containing from 1 to 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5 ethylene glycol units, to which is bonded a lysine group (preferably at its carboxylic acid moiety) which binds one or two DNP groups to the lysine at the amino group(s) of lysine. Still other linkers comprise amino acid residues (D or L) to which are bonded to A_{B}M moieties, in particular, DNP, among others at various places on amino acid residue as otherwise described herein. In another embodiment, as otherwise described herein, the amino acid has anywhere from 1-15 methylene groups separating the amino group from the acid group in providing a linker to the A_{B}M moiety.

Or another linker is according to the chemical formula:

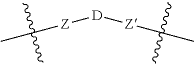

Where Z and Z′ are each independently a bond, —(CH₂)ᵢ—O, —(CH₂)ᵢ—S, —(CH₂)ᵢ'N—R,

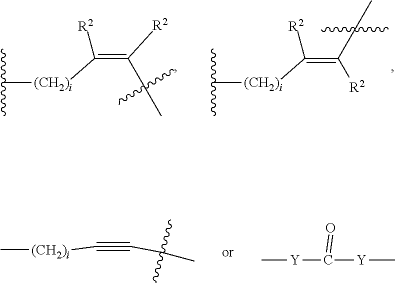

wherein said —(CH₂)ᵢ group, if present in Z or Z′, is bonded to a connector (CT), A_{B}M and/or FBM;
Each R is H, or a C₁-C₃ alkyl or alkanol group;
Each R² is independently H or a C₁-C₃ alkyl group;
Each Y is independently a bond, O, S or N—R;
Each i is independently 0 to 100, 0 to 75, 1 to 60, 1 to 55, 1 to 50, 1 to 45, 1 to 40, 2 to 35, 3 to 30, 1 to 15, 1 to 10, 1 to 8, 1 to 6, 0, 1, 2, 3, 4 or 5;

### D is

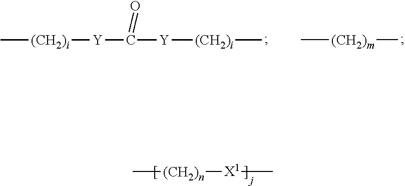

or
a bond, with the proviso that Z, Z′ and D are not each simultaneously bonds;
j is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
m′ is 1 to 100, 1 to 75, 1 to 60, 1 to 55, 1 to 50, 1 to 45, 1 to 40, 2 to 35, 3 to 30, l to 15, 1 to 10, 1 to 8, 1 to 6, 1, 2, 3, 4 or 5;
n is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5 (n is preferably 2);

### X¹ is O, S or N—R; and

R is as described above, or a pharmaceutical salt thereof.

Other linkers which are included herein include preferred linkers according to the chemical structure:

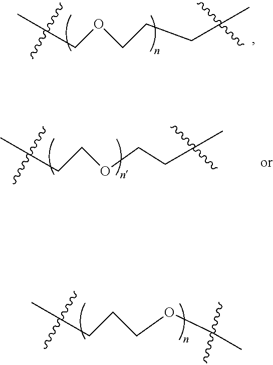

where each n and n′ is independently 1 to 25, 1 to 15, 1 to 12, 2 to 11, 2 to 10, 2 to 8, 2 to 6, 2 to 5, 2 to 4 and 2 to 3 or 1, 2, 3, 4, 5, 6, 7, or 8; and
each n″ is independently 0 to 8, often 1 to 7, or 1, 2, 3, 4, 5 or 6 (preferably 3).

Preferred linkers which include a CT group (especially a diamide CT group as otherwise described herein) connecting a first and second (e.g. a PEG) linker group include the following structures:

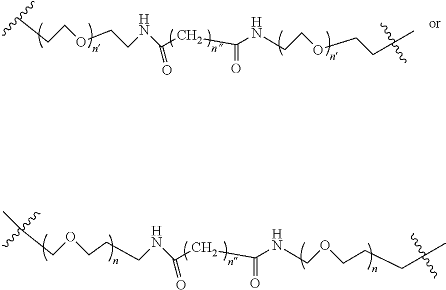

where each n and n′ is independently 1 to 25, 1 to 15, 1 to 12, 2 to 11, 2 to 10, 2 to 8, 2 to 6, 2 to 5, 2 to 4 and 2 to 3 or 1, 2, 3, 4, 5, 6, 7, or 8; and
each n″ is independently 0 to 8, often 1 to 7, or 1, 2, 3, 4, 5 or 6 (preferably 3). Noted is that each of these linkers may contain alkylene groups containing from 1 to 4 methylene groups at the distal ends of each linker group in order to facilitate connection of the linker group.

The term “connector”, symbolized in the generic formulas by (CT) is used to describe a chemical moiety which is optionally included in bifunctional compounds according to the present invention which forms from the reaction product of an activated A_{B}M-linker with a fungal binding moiety FBM moiety (which also is preferably activated) or an A_{B}M moiety with an activated linker-FBM as otherwise described herein. The connector group is often the resulting moiety which forms from the facile condensation of two or more separate chemical fragments which contain reactive groups which can provide connector groups as otherwise described to produce bifunctional or multifunctional compounds according to the present invention. It is noted that a connector may be distinguishable from a linker in that the connector is the result of a specific chemistry which is used to provide bifunctional compounds according to the present invention wherein the reaction product of these groups results in an identifiable connector group or part of a connector group which is distinguishable from the linker group, although in certain instances, the connector group is incorporated into and integral with the linker group as otherwise described herein. It is noted also that a connector group may be linked to a number of linkers to provide multifunctionality (i.e., more than one fungal binding moiety FBM moiety and/or more than one A_{B}M moiety within the same molecule. It is noted that there may be some overlap between the description of the connector group and the linker group such that the connector group is actually incorporated or forms part of the linker, especially with respect to more common connector groups such as amide groups, oxygen (ether), sulfur (thioether) or amine linkages, urea or carbonate —OC(O)O— groups as otherwise described herein. It is further noted that a connector (or linker) may be connected to A_{B}M, a linker or FBM at positions which are represented as being linked to another group using the symbol

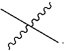

Where two or more such groups are present in a linker or connector, any of an A_{B}M, a linker or a FBM may be bonded to such a group. Where that symbol is not used, the linker may be at one or more positions of a moiety.

Common connector groups which are used in the present invention include the following chemical groups:

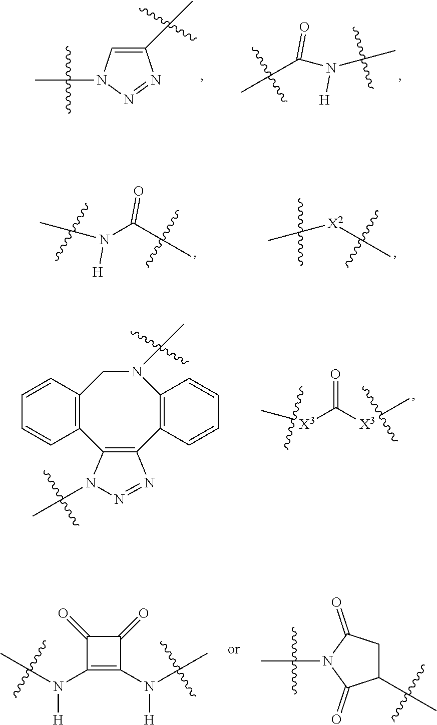

or a diamide group according to the structure:

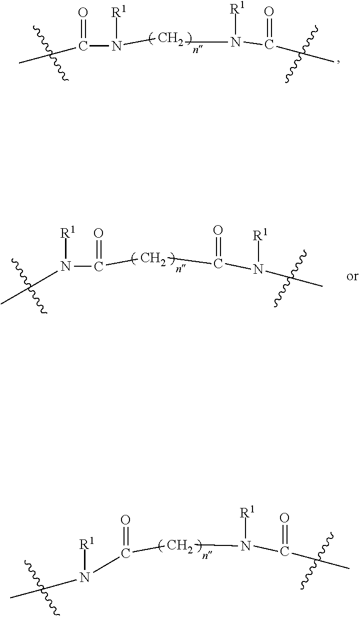

Where X² is CH₂, O, S, NR⁴, C(O), S(O), S(O)₂, —S(O)₂O, —OS(O)₂, or OS(O)₂O;
X³ is O, S, NR⁴;
R⁴ is H, a C₁-C₃ alkyl or alkanol group, or a —C(O)(C₁-C₃) group;
R¹ is H or a C₁-C₃ alkyl group (preferably H); and
n″ is independently 0 to 8, often 1 to 7, or 1, 2, 3, 4, 5 or 6 (preferably 3). The triazole group, indicated above, is a preferred connector group. It is noted that each connector may be extended with one or more methylene groups to facilitate connection to a linker group, another CT group, a FBM group or a A_{B}M/ABT group. It is noted that in certain instances, within context the diamide group may also function independently as a linker group.

It is noted that each of the above groups may be further linked to a chemical moiety which bonds two or more of the above connector groups into a multifunctional connector, thus providing complex multifunctional compounds comprising more than one A_{B}M and/or group and a number of linker groups within the multifunctional compound.

The term “alkyl” refers to a fully saturated monovalent radical containing carbon and hydrogen, and which may be cyclic, branched or a straight chain containing from 1 to 10 carbon atoms, (1, 2, 3, 4, 5, 6, 7, 8, 9 or 10), preferably 1, 2 or 3 carbon atoms. Examples of alkyl groups are methyl, ethyl, n-butyl, n-hexyl, n-heptyl, n-octyl, isopropyl, 2-methylpropyl, cyclopropyl, cyclopropylmethyl, cyclobutyl, cyclopentyl, cyclopentylethyl, cyclohexylethyl and cyclohexyl. Preferred alkyl groups are C₁-C₆ or C₁-C₃ alkyl groups. “Alkylene” (e.g., methylene) when used, refers to a fully saturated hydrocarbon which is divalent (may be linear, branched or cyclic) and which is optionally substituted. Other terms used to indicate substitutuent groups in compounds according to the present invention are as conventionally used in the art.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. ARM-F compounds according to the present invention may be administered with one or more additional anti-fungal agents or other agents which are used to treat or ameliorate the symptoms of fungal infections. Exemplary anti-fungal agents which may be coadministered in combination with one or more chimeric compounds according to the present invention include, for example, polyenes, imidazoles, triazoles, allylamines, and echinocandins, as well as miscellaneous agents, among numerous others, as otherwise described herein. In certain preferred aspects of the present invention, compounds according to the invention are co-administered with echnocandins, which are favorably administered to trigger a specific drug-induced condition by reducing the amount of glucan in the fungal cell wall and increasing the synthesis and surface exposure of chitin, making the fungus more vulnerable to inhibition by the present compounds. The result is a particularly favorable (synergistic) impact on fungal growth.

The term “blocking group”, “P_{G}” refers to a group which is introduced into a molecule by chemical modification of a functional group to obtain chemoselectivity in a subsequent chemical reaction. It plays an important role in providing precursors to chemical components which provide compounds according to the present invention. Blocking groups may be used to protect functional groups on a CT groups, a FBM group or a A_{B}M/ABT group of linker molecules in order to assemble compounds according to the present invention. Typical blocking groups are used on alcohol groups, amine groups, carbonyl groups, carboxylic acid groups, phosphate groups and alkyne groups among others.

Exemplary alcohol/hydroxyl protecting groups include acetyl (removed by acid or base), benzoyl (removed by acid or base), benzyl (removed by hydrogenolysis, β-methoxyethoxymethyl ether (MEM, removed by acid), dimethoxytrityl [bis-(4-methoxyphenyl)phenylmethyl](DMT, removed by weak acid), methoxymethyl ether (MOM, removed by acid), methoxytrityl [(4-methoxphenyl)diphenylmethyl], (MMT, Removed by acid and hydrogenolysis), p-methoxylbenzyl ether (PMB, removed by acid, hydrogenolysis, or oxidation), methylthiomethyl ether (removed by acid), pivaloyl (Piv, removed by acid, base or reductant agents. More stable than other acyl protecting groups, tetrahydropyranyl (THP, removed by acid), tetrahydrofuran (THF, removed by acid), trityl (triphenyl methyl, (Tr, removed by acid), silyl ether (e.g. trimethylsilyl or TMS, tert-butyldimethylsilyl or TBDMS, tri-iso-propylsilyloxymethyl or TOM, and triisopropylsilyl or TIPS, all removed by acid or fluoride ion such as such as NAF, TBAF (tetra-n-butylammonium fluoride, HF-Py, or HF-NEt₃); methyl ethers (removed by TMSI in DCM, MeCN or chloroform or by BBr₃ in DCM) or ethoxyethlyl ethers (removed by strong acid).

Exemplary amine-protecting groups include carbobenzyloxy (Cbz group, removed by hydrogenolysis), p-Methoxylbenzyl carbon (Moz or MeOZ group, removed by hydrogenolysis), tert-butyloxycarbonyl (BOC group, removed by concentrated strong acid or by heating at elevated temperatures), 9-Fluorenylmethyloxycarbonyl (FMOC group, removed by weak base, such as piperidine or pyridine), [2-(Trimethylsilyl)ethoxycarbonyloxy] (Teoc group), trichloroethyl chloroformate (Troc group), Benzotriazol-l-yl allyl carbonate s(Alloc group), acyl group (acetyl, benzoyl, pivaloyl, by treatment with base), benzyl (Bn groups, removed by hydrogenolysis), carbamate, removed by acid and mild heating, p-methoxybenzyl (PMB, removed by hydrogenolysis), 3,4-dimethoxybenzyl (DMPM, removed by hydrogenolysis), p-methoxyphenyl (PMP group, removed by ammonium cerium IV nitrate or CAN); tosyl (Ts group removed by concentrated acid and reducing agents, other sulfonamides, Mesyl, Nosyl & Nps groups, removed by samarium iodide, tributyl tin hydride, azido group and phthalimide.

Exemplary carbonyl protecting groups include acyclical and cyclical acetals and ketals (removed by acid), acylals (rembyed by Lewis acids) and dithianes (removed by metal salts or oxidizing agents).

Exemplary carboxylic acid protecting groups include methyl esters (removed by acid or base), benzyl esters (removed by hydrogenolysis), tert-butyl esters (removed by acid, base and reductants), esters of 2,6-disubstituted phenols (e.g. 2,6-dimethylphenol, 2,6-diisopropylphenol, 2,6-di-tert-butylphenol, removed at room temperature by DBU-catalyzed methanolysis under high-pressure conditions, silyl esters (removed by acid, base and organometallic reagents), orthoesters (removed by mild aqueous acid), oxazoline (removed by strong hot acid (pH<1, T>100° C.) or strong hot alkali (pH>12, T>100° C.)).

Exemplary phosphate group protecting groups including cyanoethyl (removed by weak base) and methyl (removed by strong nucleophiles, e.g. thiophenol/TEA).

Exemplary terminal alkyne protecting groups include propargyl alcohols and silyl groups.

The ARM-F containing pharmaceutical compositions of the present invention may be formulated in a conventional manner using one or more pharmaceutically acceptable carriers and may also be administered in immediate, early release or controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. In certain preferred aspects of the invention, the topical cream or lotion may be used prophylatically to prevent infection when applied topically in areas prone toward virus infection. In additional aspects, the compounds according to the present invention may be coated onto the inner surface of a condom and utilized to reduce the likelihood of infection during sexual activity.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and disease treated, the particular mode of administration. Preferably, the compositions should be formulated to contain between about 0.05 milligram to about 1 to several grams, more preferably about 1 milligram to about 750 milligrams, and even more preferably about 10 milligrams to about 500-600 milligrams of active ingredient, alone or in combination with at least one other ARM-F compound according to the present invention or other anti-cancer agent which may be used to treat cancer or a secondary effect or condition thereof.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject (e.g. a male or female human) suffering from cancer can be treated by administering to the patient (subject) air effective amount of the ARM-F compound according to the present invention including pharmaceutically acceptable salts, solvates or polymorphs, thereof optionally in a pharmaceutically acceptable carrier or diluent, either alone, or in combination with other known pharmaceutical agents, preferably agents which can assist in treating cancer and/or secondary effects of cancer or ameliorate the secondary effects and conditions associated with cancer, including metastasis of cancer. This treatment can also be administered in conjunction with other conventional cancer therapies, including radiation therapy.

These compounds can be administered by any appropriate route, for example, orally, parenterally, intravenously, intradermally, subcutaneously, or topically, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably about 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, preferably about 5 to 500-600 mg or more of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a giidant such as colloidal silicon dioxide; a sweetening agent such as sucrose or saccharin; or a flavoring agent such as peppermint, methyl, salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as other anticancer agent, anti-HIV agents, antibiotics, antifungals, anti-inflammatories, or antiviral compounds. In certain preferred aspects of the invention, one or more ARM-F compounds according to the present invention are coadministered with another anticancer agent and/or another bioactive agent, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### General Chemical Synthesis

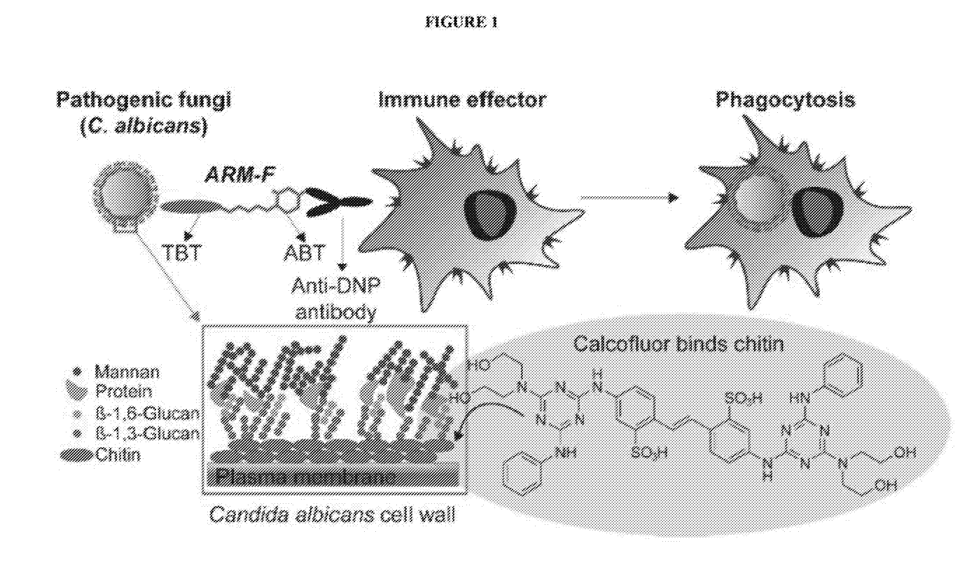

All ARM F derivatives may be synthesized by performing a highly general, optimized nucleophillic substitution reaction sequence carried out between the ABT variant of choice and a core intermediate consisting of a substituted stilbene moiety to which triazine moieties are covalently bonded on each of the phenyl groups. The triazine moieties are functionalized to accommodate a linker-ABT group as indicated (See FIG. 1, Schemes 1-3). Other compounds which are disclosed are synthesized by analogy using standard methods which are readily available in the art. The compounds may be symmetrical or asymmetrical, depending upon the biological activity desired. These compounds, readily prepared, are biologically active.

The following specific compounds were prepared pursuant to the chemical synthetic schemes which are presented in attached FIG. 1, Schemes 1-3. These compounds are:

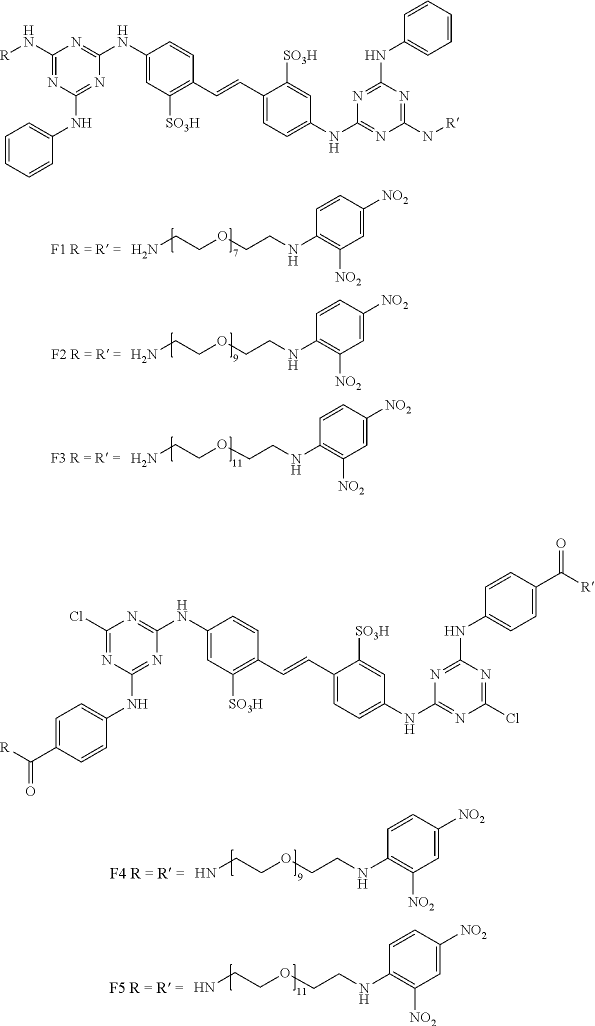

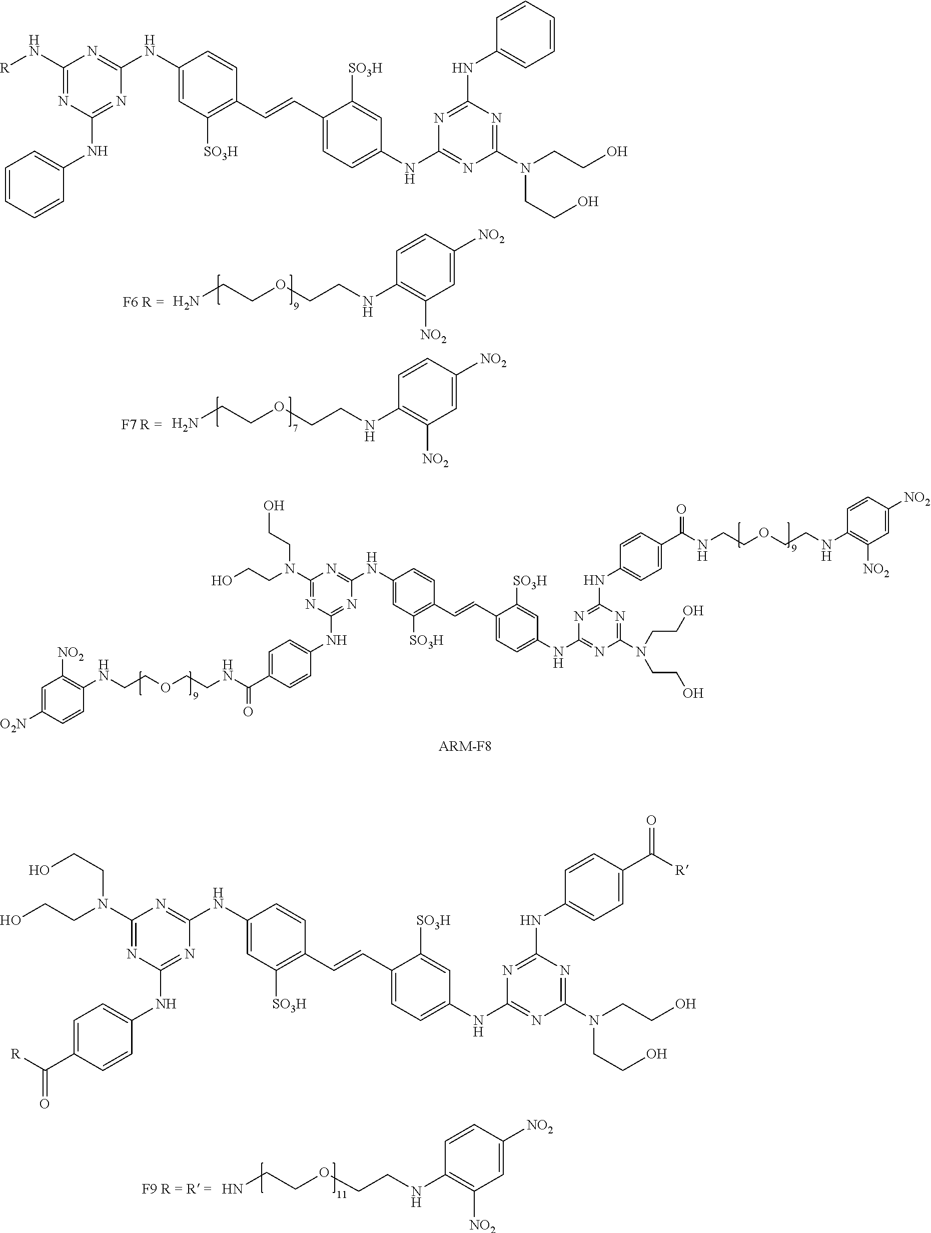

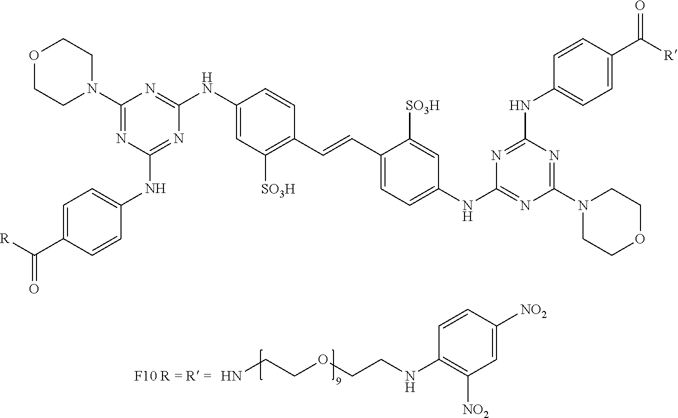

The design of candidate antifungal ARM (called ARM-F) started with calcofluor white as the TBT (FIG. 5, Scheme 4). Calcoffuor white is a well-studied fungal whitening agent known to possess high specificity for chitin, and antifungal properties at high doses.^{[11} Because ARM-F needs to bind simultaneously to fungal chitin and anti-DNP antibodies, the inventors sought a site for linker attachment on calcofluor white that would not compromise chitin-binding activity. The inventors therefore prepared three candidate ARM-F derivatives: compound 6 resulted from the replacement of diethanolamine moieties of calcofluor whereas 7 and 8 were obtained by linker incorporation into aniline functionalities.

Synthesis of compounds 6-8 proved relatively straightforward and followed the three-step route illustrated in FIGS. 2-5. Pursuant to the approach presented in FIG. 5, Scheme 4, trans-4,4′-diaminostilbene 2,2′-disulfonic acid (1) was first allowed to react with cyanuric chloride followed by gradual substitution on the triazine core of 2 with amines 3 or 4 (FIG. 5, Scheme 4). The degree of substitution was fine-tuned by controlling temperature and pH of the reaction, thus allowing the selective incorporation of ABT into the stilbene scaffold.^{[7], [3]}

Particular experimental details related to the chemical synthesis are presented in the Further experimental details section which is presented hereinbelow. This section is to be viewed as providing exemplary approaches to synthesis of chemical compounds according to the present invention and is not to be construed as limiting the invention in any way. The skilled practitioner will clearly recognize that additional compounds are readily synthesized using similar procedures or by analogy using methods which are generally known and readily available in the art.

### Biological Activity

### Assessment of Antibody-Recruiting Activity of ARM-Fs

The antibody-recruiting activity of the prepared derivatives was evaluated on Candida albicans using flow cytometry, described in the experimental section below. The compound F8 was found to recruit the anti-DNP antibodies in a dose-dependent manner, with the highest activity reached at 1 μM concentration. A bell-shaped, autoinhibitory dose-response curve was observed at the concentrations of F8 greater than 1.25 μM which is consistent with its mode of action through the formation of tertiary complexes (FIG. 6). See Douglas, et al., J. Am. Chem. Soc., 2013, 135, 6092-6099.

Next, the ability of compound F8 to recruit the anti-DNP antibodies in the presence of Caspofungin (Cancidas®) was evaluated. The carbohydride polymers glucan and chitin are two interrelated elements of fungal cell wall which are critical to maintaining its physical integrity. We envisaged that the combination of ARM-F agent with the (1→3)β-D-glucan synthesis inhibitor Caspofungin would result in a synergistic effect through (i) the morphological changes of fungal cell wall leading to higher surface exposure of chitin and (ii) enhanced chitin synthesis. Indeed, the antibody-recruitment was enhanced in the presence of Caspofungin, with the maximum observed at 1 ng/mL concentration of Caspofungin (FIG. 7).

### Representative Procedure for Antibody-Recruiting Assays

Candida albicans cultures were maintained on Sabouraud dextrose agar (SDA) and yeast cell cultures were grown at 30° C. in Sabouraud dextrose broth with shaking at 225 rpm overnight (OD 1.1-1.3). For the assays, 10⁵ cells were taken into 50 μL assay medium (5% BSA in PBS 1x) and treated with a fixed concentration of the indicated ARM-F compound from 100x DMSO stocks followed by imminent addition of 50 μL 500x stock solution of anti-DNP-biotine-xx conjugate antibodies in assay medium. Cells were allowed to incubate for 30 min at r.t., then washed twice with 500 μL assay medium followed by addition of 50 μL 500x stock solution of streptavidin-AlexaFluor 647 conjugate and propidium iodide. Negative control tubes contained streptavidin +/− anti-DNP antibodies in the absence of ARM-F compound. The cells were allowed to incubate for another 30 min, washed with 500 μL assay medium and antibody recruiting to the cell surface was evaluated via flow cytometry by measuring increasing cell counts in the FL-4 channel negative for FL-3.

The synergistic experiments with Caspofungin were performed as described above, Save that yeast cell cultures were grown in Sabouraud dextrose broth supplemented with a fixed concentration of Caspofungin acetate from DMSO stock solutions.

### Further/Alternative Experiments

A flow-cytometry based assay was developed to assess the antibody recruitment ability of candidate ARM-Fs to isolates of C. albicans. We chose this organism for our studies because it is both a known human pathogen, and readily tractable for experimental manipulations.^{[13]} Ternary complexes formed from ARM-F, fungal cells and anti-DNP antibodies were detected using an anti-DNP secondary antibody conjugated to Alexa Fluor 647 fluorophore. Among the ARM-F candidates prepared,^{[10a]} compound 8 demonstrated the highest antibody-recruiting activity with 35-45% of C. albicans cells exhibiting AF647 fluorescence (FIG. 8A), resistance to echinocandins.^{[17] At} 1 μM concentration of 8, antibody recruitment positively correlates with caspofungin dosage. At 1.25 ng/ml treatment with caspofungin, 90% of the fungal cells recruited antibodies and were alive, while higher concentrations resulted in reduced viability (FIG. 9C). In dose ranging studies, 1 μM of 8 elicited a maximum response in caspofungin treated cells. A hundredfold reduced dose (10 nM) was able to elicit antibody recruitment comparable to the maximum possible in the non-caspofungin model (FIG. 9D). Compound 9 did not recruit antibodies to caspofungin-treated C. albicans cells, ruling out artefactual “sticky” effects of caspofungin resulting in enhanced recruitment by 8 (FIG. 9D). These observations establish that treatment of fungi with sub-inhibitory concentrations of caspofungin enhances both the functionality and potency of 8. These results also suggest that combinations of echinocandins and ARM-F may prove synergistic clinically.

Taken together, these experiments suggest that ARM-F (8) binds to fungal chitin in a dose-dependent manner and recruits antibodies to the surface of fungal cells. These conclusions prompted us to perform functional assays evaluating the ability of 8 to mediate immune clearance of fungal pathogens by redirecting human immune effector cells.

In the physiological state, neutrophils mediate immune clearance of antibody-opsonized fungal cells.^{[18], [19]} To mimic this mechanism, we developed a functional assay to analyse ARM-F mediated opsonophagocytosis of fungal cells. The assay is based on two color flow cytometry and operates on the principle of differentiating phagocytosed fungal cells (intracellular) from non-phagocytosed cells (extracellular).^{[20]} Thus, FITC-labeled C. albicans were treated with ARM-F (8) and co-incubated with human neutrophils in the presence of anti-DNP antibodies After completion of incubation, polyclonal rabbit anti-Candida antibodies in combination with AF647-labeled anti-rabbit immunoglobulin G (IgG) were used to fluorescently tag the free non-phagocytosed fungal cells. Upon selective osmotic disruption of neutrophils, the single (FITC only) labeled phagocytosed cells were released and could be easily differentiated from the dual (FITC and AF647) labeled non-phagocytosed fungal cells by flow cytometry (FIGS. 4A and B). As anticipated, the treatment with ARM-F (8) induced a drastic increase in number of phagocytosed fungal cells compared to the untreated cells. ARM-F (8) induced a dose-dependent phagocytosis of C. albicans cells with the maximal response at 10 μM again showing the bell-shaped curve characteristic of ternary complexes (FIG. 10C).

Particular experimental details related to the biological activity are presented in the Further experimental details section which is presented hereinbelow.

### Conclusion

In conclusion, the inventors discovered novel bifunctional small molecules that can target pathogenic fungi toward immune-based clearance. The high-affinity molecule ARM-F (8) demonstrated a specific, dose-dependent binding to fungal chitin, recruited anti-DNP antibodies, and consequently mediated dose-dependent phagocytosis of the fungal cells by human immune cells. The utilization of the host's own immune system as opposed to cytotoxic agents for the elimination of pathogens makes this approach desirable from a therapeutic standpoint. The inventors posit that ARM-F (8) singly or in combination with currently marketed antifungals promises to be an effective antifungal immunotherapy for combating C. albicans and other fungal infections. Enhanced levels of ARM-F (8) activity in the C. albicans model following pre-treatment with the echinocandin antifungal caspofungin also suggests potential therapeutic utility against antifungal-resistant infections.

### REFERENCES

[1] G. D. Brown, D. W. Denning, N. A. R. Gow, S. M. Levitz, M. G. Netea, T. C. White, Sci. Transl. Med. 2012, 4, 165rv13.

[2] a) J. Kriengkauykiat, J. I. Ito, S. S. Dadwal, Clin. Epidemiol. 2001, 3, 175-191; b) J. Delaloye, T. Calandra, Virulence, 161-169; c) G. S. Martin, D. M. Mannino, S. Eaton, M. Moss, N. Engl. J. Med. 2003, 348, 1546-1554.

[3] P. Vandeputte, S. Ferrari, A. T. Coste, Int. J. Microbiol. 2012, Article ID 713687, 26 p.

[4] A. H. Fairlamb, N. A. R. Gow, K. R. Matthews, A. P. Waters, Nat. Microbiol. 2016, 16092.

[5] L. Nimrichter, M. L. Rodrigues, E. G. Rodrigues, L. R. Travassos, Microbes Infect. 2005, 7, 789-798.

[6] B. J. Harrington, G. L. Hageage, Jr., Lab. Med. 2003, 34, 361-367.

[7] T. Meehan, Q. Ong, Patent U.S. 2015/0037396 A1, 2015; [8] W Herth, E. Schnepf, Protoplasma 1980, 105, 129-133.

[9] a) E. Ortega, M. Kostovetzky, C. Larralde, Mol. Immunol. 1984, 21, 883-888; b) S. Jormalainen, O. Mäkelä, Eur. J. Immunol. 1971, 1, 471-478; c) K. Karjalainen, O. Mäkelä, Eur. J. Immunol. 1976, 6, 88-93; d) F. S. Farah, Immunology 1978, 25, 217-226.

[10] For selected recent publications, see: a) A. F. Rullo, K. J. Fitzgerald, V. Muthusamy, M. Liu, C. Yuan, M. Huang, M. Kim, A. E. Cho, D. A. Spiegel, Angew. Chem. 2016, 128, 3706-3710; Angew. Chem. Int. Ed. 2016, 7, 3642-3646; b) C. G. Parker, K. M. Dahlgren, R. N. Tao, D. T. Li, E. F. Douglass, T. Shoda, N. Jawanda, K. A. Spasov, S. Lee, N. Zhou, R. A. Domaoal, R. E. Sutton, K. S. Anderson, M. Krystal, W. L. Jorgenson, D. A. Spiegel, Chem. Sci. 2014, 5, 2311-2317; c) C. E. Jakobsche, C. G. Parker, R. N. Tao, M. D. Kolesnikova, E. F. Douglass, Jr., D. A. Spiegel, ACS Chem. Biol. 2013, 8, 2404-2411; (d) C. G. Parker, R. A. Domaoal, K. S. Anderson, D. A. Spiegel, J. Am. Chem. Soc. 2009, 131, 16392-16394.

[11] J. M. Kingbury, J. Heitman, S. R. Pinnell PloS ONE 2012, 7, e39405.

[12] a) A. Saeed, G. Shabir, I Batool, J. Fluoresc. 2014, 24, 1119-1127; b) M. Wan, S. Zhou, P. Jiao, C. Cao, J. Guo, J. Fluoresc. 2013, 23, 1099-1105; c) M. Okuom, M. Wilson, J. Groathouse, J. Lee, D. Symonsbergen, C. Gustafson, M. Trauernicht, H. Barcena, C. Reicks, S. Sikich, R. Burks, A. Holmes, Int. J. Org. Chem. 2013, 3, 256-261.

[13] C. F. Urban, U. Reichard, V. Brinkmann, A. Zychlinsky, Cell Microbiol. 2006, 8, 668-676.

[14] E. F. Douglass, Jr., C. J. Miller, G. Sparer, H. Shapiro, D. A. Spiegel, J. Am. Chem. Soc. 2013, 135, 6092-6099.

[15] R. Schekman, V. Brawley, Proc. Natl. Acad. Sci. U.S.A. 1979, 76, 645-649.

[16] M. Schmidt, Microbiology 2004, 150, 3253-3260.

[17] a) L. A. Walker, N. A. Gow, C. A. Munro, Antimicrob. Agents Chemother. 2013, 57, 146-154; b) K. K. Lee, D. M. Maccalum, M. D. Jakobsen, L. A. Walker, F. C. Odds, N. A. Gow, C. A. Munro, Antimicrob. Agents Chemother. 2012, 56, 208-217; c) L. A. Walker, C. A. Munro, I. de Bruijn, M. D. Lenardon, A. McKinnon, N. A. Gow, PLoS Pathog. 2008, 4, e1000040.

[18] C. S. Farah, S. Elahi, G. Pang, T. Gotjamanos, G. J. Seymour, R. L. Clancy, R. B. Ashman, Infect. Immun. 2001, 69, 6110-6118.

[19] a) N. Brouwer, K. M. Dolman, M. van Houdt, M. Sta, D. Roos, T. W. Kuijpers, J. Immunol. 2008, 180, 4124-4132; b) M. Wellington, K. Dolan, C. G. Haidaris, FEMS Immunol. Med. Microbiol. 2007, 51, 70-83.

[20] For in vitro phagocytosis studies, see: a) K. H. Kim, J. Y. Seoh, S. J. Cho, J. Korean Med. Sci. 2015, 30, 145-150; b) C. Cameiro, C. Vaz, J. Carvalho-Pereira, C. Pais, P. J. Sampaio, J. Microbiol. Methods 2014, 101, 56-62; c) K. Dementhon, S. El-Kirat-Chatel, T. Noël, PLoS One 2012, 7, e32621; d) S. Busetto, E. Trevisan, P. Patriarca, R. Menegazzi, Cytometry A 2004, 58, 201-206.

### Further Experimental Details

### Abbreviations

ABT, antibody-binding terminus; Ac, acetate; aq., aqueous; ARM, antibody-recruiting small molecule; Boc, tert-butyloxycarbonyl; BSA, bovine serum albumin; tBu, tert-butyl; DIPEA, N,N-diisopropylethylamine; DMF, N,N-dimethylformamide; PMSO, dimethyl sulfoxide; ESI, electrospray ionization; Et, ethyl; FACS, fluorescence activated cell sorting; FITC, fluorescein isocyanate; HBTU, 2-(1H-benzotriazol-l-yl)-1,1,3,3-tetramethyluronium hexafluorophosphate; HRP, horseradish peroxidase; KLH, keyhole limpet hemocyanin; m/z, mass-to-charge ratio; Me, methyl; MEK, methyl ethyl ketone; Ms, mesyl, methansulfonyl; PBS, phosphate-buffered saline; ppm, part per million; prep., preparative; quant., quantitative; r.t., room temperature; susp., suspension; TEA, triethylamine; THF, tetrahydrofuran; Ts, tosyl, p-toluenesulfonyl; YPD, yeast extract peptone dextrose.

### Chemical Synthesis

I. General Procedure

All the reactions were carried out under argon or nitrogen atmosphere with dry solvents under anhydrous conditions, unless otherwise stated. Commercially available reagents were used without further purification, unless otherwise stated. Flash chromatography on silica gel was performed using Isco Teledyne CombiFlash Rf system. High-performance liquid chromatography was carried out using Waters 996 PDA HPLC system equipped with a SunFire™ 5 μL 4.6×150 mm C18 column. Isolated yields are given, unless otherwise noted. NMR spectra were recorded on either Agilent DD2 500 MHz or Agilent DD2 600 MHz NMR spectrometers and treated with MestReNova MestreLab software. NMR spectra were calibrated using the residual peaks of deuterated solvents as internal referenee (CDCl₃: ¹H 7.26, ¹³C 77.0 ppm; (CD₃)₂SO: ¹H 2.50, ¹³C 39.5 ppm). Where noted, coupling constants are given in Hz. Multiplicities are referred to as: s—singlet, d—doublet, t—triplet, q—quartet, br—broad signal, m—multiplet. IR spectra were recorded on Thermo Nicolet 6700 FT-IR instrument; frequencies are given in reciprocal centimeters (cm⁻¹) and only selected absorbance is reported. LC-MS analyses were recorded using Waters UPLC/MS instrument equipped with a 2.1×50 mm C18 column and dual atmospheric pressure chemical ionization (API)/electrospray (ESI) mass spectrometry detector.

II. Experimental Protocols and Characterization Data
IIa. Synthesis of Linkers and AST Functionalization

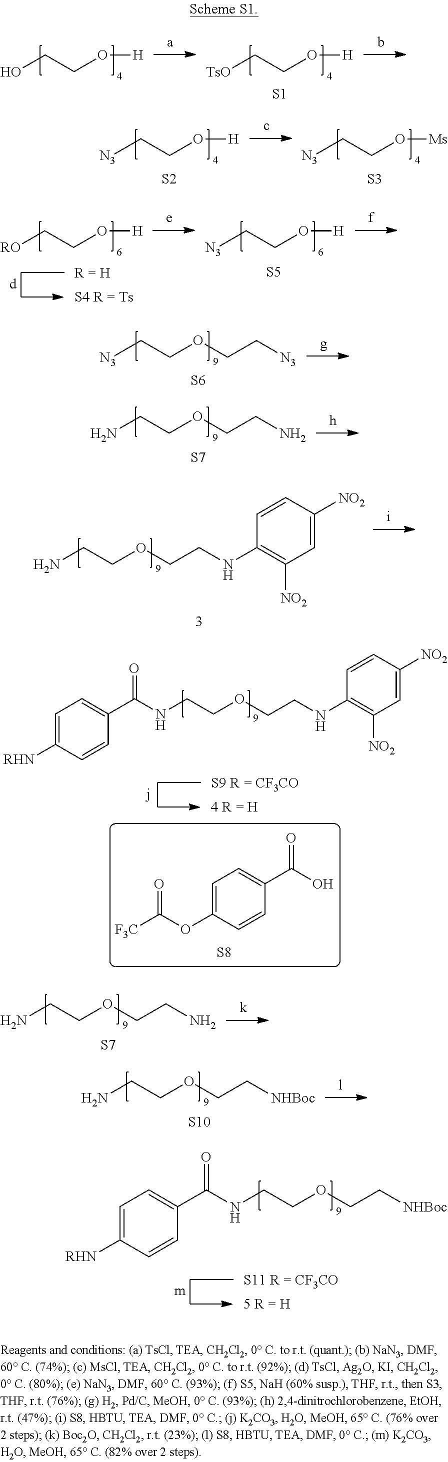

Tetraethylene Glycol p-toluensulfonic Acid Ester S1 (C₁₅H₂₄O—S, 348,41)

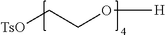

To a solution of tetraethylene glycol (101.90 g, 524 mmol, 10 equiv) in CH₂Cl₂ (100 mL) was added tosyl chloride (10.01 g, 52.4 mmol) followed by TEA (11 mL, 7.96 g, 78.7 mmol, 1.5 equiv) at 0° C. The mixture was stirred at r.t. overnight, then poured into a separatory funnel and washed (3×100 mL) with water. The combined organic layers were dried (Na₂SO₄), filtered and concentrated in vacuo to afford S1 (18.2 g, 52.5 mmol, quant) as a colorless oil. The spectroscopic data of S1 matched well those reported in literature.^{1 1}H NMR (CDCl₃, 500 MHz): δ 7.77 (d, J=8.2 Hz, 2H), 7.32 (d, J=8.1 Hz, 2H), 4.12 (dt, J₁=9.4 Hz, J₂=4.7 Hz, 2H), 3.72-3.53 (m, 14H), 2.42 (s, 3H). MS (ESI) [M+H]⁺: m/z 349. ¹Heller, K.; Ochtrop, P.; Albers, M. F.; Zauner, F. B.; Itzen, A.; Hedberd, C. Angew. Chem. Int. Ed. 2015, 54; 10327-10330.
2-(2-(2-(2-azidoethoxy)ethoxy)ethoxy)ethanol S2 (C₈H₁₇N₃O₄, 219.24)

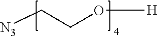

Sodium azide (4.10 g, 63.0 mmol, 1.2 equiv) was added to a solution of S1 (18.2 g, 52.5 mmol) in DMF (200 mL) and the mixture was stirred at 60° C. overnight. Upon completion, the volume of DMF was reduced in vacuo to a ⅓ of its initial volume, diluted with water (100 mL) and extracted with EtOAc (3×100 mL). The combined organic layers were dried (Na₂SO₄), filtered and concentrated in vacuo to afford S2 (8.51 g, 38.8 mmol, 74%) as a pale yellow oil. The spectroscopic data of S2 matched well those reported in literature.^{2 1}H NMR (CDCl₃, 500 MHz): δ 3.65-3.61 (m, 2H), 3.58 (s, 10H), 3.54-3.50 (m, 2H), 3.31 (t, J=4.7 Hz, 2H), 2.89 (br s, 1H). MS (ESI) [M+H]⁺: m/z 220. ²Besenius, P.; Cormack, P. A. G.; Ludlow, R. F.; Otto, S.; Sherrington, D. C. Chem. Commun. 2008, 24, 2809-2811.
2-(2-(2-(2-azidoethoxy)ethoxy)ethoxy)ethyl methanesulfonate S3 (C₁₉H₁₉N₃O₆S, 297.33).

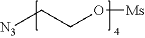

An oven-dried Schlenk tube equipped with a magnetic stirring bar and an argon inlet adapter was charged with a solution of S2 (5.04 g, 23.0 mmol) and TEA (6.4 mL, 4.65 g, 46.0, 2 equiv) in CH₂Cl₂ (100 mL). Mesyl chloride (2.31 mL, 3.42 g, 29.9 mmol, 1.3 equiv) was added dropwise at 0° C. and the mixture was stirred for an additional 10 min. at this temperature. The ice bath was removed and the mixture was stirred for 40 min at r.t., then all volatiles were removed in vacuo and the residue was purified by flash chromatography (hexanes/EtOAc 1:0 to 1:1) to afford S3 (6.31 g, 21.2 mmol, 92%) as a pale yellow oil. The spectroscopic data of S3 matched well those reported in literature.^{3 1}H NMR (CDCl₃, 500 MHz): δ4.38 (dd, J₁=5.3 Hz, J₂=3.7 Hz, 2H), 3.77 (dd, J₁=10.1 Hz, J₂=5.6 Hz, 2H), 3.69-3.64 (m, 10H), 3.39 (t, J=5.0 Hz, 2H), 3.07 (s, 3H). MS (ESI) [M+Na]*: m/z 320. ³Svedhem, S.; Hollander, C. A.; Shi, J.; Konradsson, P.; Liedberg, B.; Svensson, S. C. T. J. Org. Chem. 2001, 66, 4494-4503.
Hexaethylene glycol p-toluenesulfonic acid ester S4 (C₁₉H₃₂O₉S, 436.52)

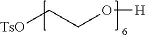

To a solution of hexaethylene glycol (2.99 g, 10.6 mmol) in CH₂Cl₂ (100 mL) were successively added tosyl chloride (2.22 g, 11.6 mmol, 1.1 equiv), silver (I) oxide (3.68 g, 15.9 mmol, 1.5 equiv) and potassium iodide (0.352 g, 0.212 mmol, 0.2 equiv) at 0° C. The mixture was stirred for 25 min at this temperature, then filtered through a pad of Celite® (ca. 1 cm), which was then rinsed with methanol. Upon removing all volatiles in vacuo, the residue was purified by flash chromatography (CH₂Cl₂/MeOH 1:0 to 9:1) to afford S4 (3.70 g, 8.48 mmol, 80%) as a colorless oil. The spectroscopic data of S4 matched well those reported in literature.^{4 1}H NMR (CDCl₃, 500 MHz): δ7.79 (d, J=7.9 Hz, 2H), 7.34 (d, J=7.9 Hz, 2H), 4.16 (t, J=4.7 Hz, 2H), 3.76-3.52 (m, 22H), 2.44 (s, 3H), MS (ESI) [M+Na]*: m/z 459. ⁴Bouzide, A.; Sauve, G. Org. Lett. 2002, 2329-2332.
2-(2-(2-(2-(2-(2-azidoethoxy)ethoxy)ethoxy)ethoxy)ethoxy)ethanol S5 (C₁₂H₂₅N₃O₆, 307.34)

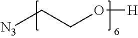

Sodium azide (1.6 g, 24.6 mmol, 1.2 equiv) was added to a solution of S4 (8.94 g, 20.5 mmol) in DMF (80 mL) and the mixture was stirred at 60° C. overnight. Upon completion, the volatiles were removed in vacuo and the residue was purified by flash chromatography (EtOAc/MeOH 9:1) to afford S5 as a pale yellow oil (5.84 g, 19.0 mmol, 93%). The spectroscopic data of S5 matched well those reported in literature.^{5 1}H NMR (CDCl₃, 500 MHz): δ 3.71 (m, 2H), 3.67 (br s, 18H), 3.61 (t, J=3.7 Hz, 2H), 3.38 (t, J=5.1 Hz, 2H), 2.50 (br s, 1H). MS (ESI) [M+H]⁺: m/z 308. ⁵Abronina, P. I.; Zinln, A. I.; Orlova, A. V.; Sedinkin, S. L.; kononov, L. O. Tetrahedron Lett, 2013, 54, 4533-4535.

1-(2-(2-(2-(2-(2-(2-(2-(2-(2-azidoethoxy)ethoxy)ethoxy)ethoxy)ethoxy)ethoxy)ethoxy)ethoxy)ethoxy)-2-azidoethane S6 (C₂₀H₄₀N₆O₉, 508.57)

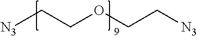

An oven-dried Schlenk tube equipped with a magnetic stirring bar and an argon inlet adapter was charged with a solution of S5 (1.91 g, 6.21 mmol) in THF (24 mL). Sodium hydride (60% susp. in mineral oil, 0.621 g, 15.5 mmol, 2.5 equiv) was added in a single portion and the mixture was stirred for 45 min at r.t. following by a dropwise addition of a solution of S3 (2.77 g, 9.32 mmol, 1.5 equiv) in THF (12 mL). The mixture was stirred for 24 h at r.t., then the solvent was removed in vacuo and the residue was purified by flash chromatography (CH₂Cl₂/EtOAc 9:1 to 1:1) to afford S6 as a pale yellow oil (2.40 g, 4.72 mmol, 76%). The spectroscopic data of S6 matched well those reported in literature.^{6 1}H NMR (CDCl₃, 500 MHz): δ 3.64 (br s, 36H), 3.39 (t, J=5.1 Hz, 4H). MS (ESI) [M+H]⁺: m/z 509. ⁶Iyer, S. S.; Anderson, A. S.; Reed, S.; Swanson, B.; Schmidt, J. G. Tetrahedron Lett. 2004, 45, 4285-4288.
(2-(2-(2-(2-(2-(2-(2-(2-(2-(2-aminoethoxy)ethoxy)ethoxy)ethoxy)ethoxy)ethoxy)ethoxy)ethoxy)ethoxy)-ethanamine S7 (C₂₀H₄₄N₂O₉, 456.57)

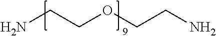

Palladium on carbon (10 wt. %, 150 mg) was added to a solution of S6 (2.40 g, 4.72 mmol) in methanol (50 mL) at 0° C. and the mixture was stirred under 1 atm H₂ at 0° C. overnight. Upon completion, the mixture was filtered through a pad of Celite® (ca. 1 cm), which was then rinsed with methanol. The solvent was removed in vacuo to afford S7 as a clear oil (2.00 g, 4.38 mmol, 93%) which was used in the next step without further purification. The spectroscopic data of S7 matched well those reported in literature.^{7 1}H NMR (CDCl₃, 500 MHz): δ 3.64 (s, 32H), 3.50 (t, J=5.0, 4H), 2.85 (t, J=5.2 Hz, 4H), 1.28 (br s, 4H). MS (ESI) [M+H]⁺: m/z 457. ⁷Bandyopadhyay, P., Bandyopadhyay, P., Regen, S. L. J. Am. Chem. Soc. 2002, 124, 11254-11255.
N-(2-(2-(2-(2-(2-(2-(2-(2-(2-(2-aminoethoxy)ethoxy)ethoxy)ethoxy)ethoxy)ethoxy)ethoxy)ethoxy)ethoxy)ethyl)-2,4-dinitrobenzenamine 3 (C₂₆H₄₆N₄O₁₃, 622.66)

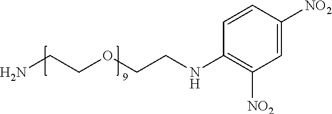

A solution of 2,4-dinitrochlorobenzene (0.9 g, 4.44 mmol, 1.05 equiv) in absolute ethanol (20 mL) was dropwise added to a solution of S7 (1.90, 4.16 mmol) in absolute ethanol (10 mL) over 10 h via a syringe pump. The mixture was stirred for another 2 h at r.t., then the solvent was removed and the residue was by purified by flash chromatography (CH₂Cl₂/MeOH 1:0 to 95:5) to afford 3 as a dark yellow oil (1.21 g, 1.94 mmol, 47%). IR (neat): v 2876, 1621, 1336, 1098 cm⁻¹. ¹H NMR (CDCl₃, 500 MHz): δ 9.14 (d, J=2.5 Hz, 1H), 8.8 (s, 1H), 8.27 (dd, J₁=9.5 Hz, J₂=2.4 Hz, 1H), 7.92 (br s, 1H), 7.00 (d, J=9.5 Hz, 1H), 3.94 (t, J=4.7 Hz, 2H), 3.85 (t, J=5.2 Hz, 2H), 3.78-3.59 (m, 35H), 3.17 (t, J=4.3 Hz, 2H). ¹³C NMR (CDCl₃, 125 MHz): δ 148.4, 136.0, 130.3, 124.3, 114.2, 70.7, 70.6, 70.5, 70.4, 70.3, 70.2, 70.1, 70.0, 69.8, 69.7, 69.6, 68.6, 66.8, 43.3, 40.6. HRMS (ESI): calcd for C₂₆H₄₇N₄O₁₃ [M+H]⁺ m/z 623.3139, found 623.2972.
4-(2,2,2-trifluoroacetamido)-N-(2-(2-(2-(2-(2-(2-(2-(2-(2-(2-(2,4-dinitrophenylamino)ethoxy)ethoxy) ethoxy)ethoxy)ethoxy)ethoxy)ethoxy)ethoxy)ethoxy)ethyl)benzamide S9 (C₃₅H₅₀F₃N₅O₁₅, 837.79)

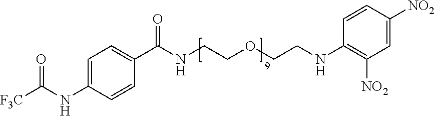

To a solution of 3 (0.636 g, 1.02 mmol) in DMF (35 mL) were successively added 4-(trifluoroacetamido)benzoic acid (S8) (0.238 g, 1.02 mmol, 1 equiv), HBTU (0.456 g, 1.23 mmol, 1.2 equiv) and TEA (213 μL, 01.55 g, 1.53 mmol, 1.5 equiv) at 0° C. The mixture was stirred for 1 h at this temperature, then filtered through pad of silica (ca. 1 cm) and concentrated in vacuo. The resultant material was used in the next step without further purification.
N-(2-(2-(2-(2-(2-(2-(2-(2-(2-(2-(2,4-dinitrophenylamino)ethoxy)ethoxy)ethoxy)ethoxy)ethoxy) ethoxy)ethoxy)ethoxy)ethoxy)ethyl)-4-aminobenzamide 4 (C₃₃H₅₁N₅O₁₄, 741.48)

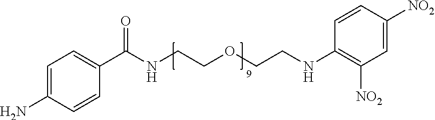

Potassium carbonate (0.423 g, 3.06 mmol, 3 equiv) was addted to a solution of S9 in methanol (75 mL) and water (25 mL). The mixture was stirred at 65° C. overnight, then conentrated in vacuo and purified by flash chromatography (CH₂Cl₂/MeOH 1:0 to 9:1)to afford 4 as a dark orange oil (0.575 g, 0.77 mmol, 76% over 2 steps). IR (neat): v 3355, 2873, 1619, 1334, 1088 cm⁻¹. ¹H NMR (CDCl₃, 500 MHz): δ 9.13-9.10 (m, 1H), 8.80-8.75 (m, 1H), 8.24 (dd, J₁=8.9 Hz, J₂=1.8 Hz, 1H), 7.61 (d, J=8.4 Hz, 2H), 6.94 (d, J=9.5 Hz, 1H), 6.64 (d, J=8.4 Hz, 2H), 4.11 (br s, 2H), 3.81 (t, J=4.7 Hz, 2H), 3.71-3.53 (m, 36H). ¹³C NMR (CDCl₃, 125 MHz): δ 167.2, 149.8, 148.4, 136.0, 130.2, 128.7, 124.2, 123.8, 114.1, 114.0, 70.7, 70.6, 70.5, 70.4, 70.1, 70.0 68.5, 43.2, 39.5. HRMS (ESI): calcd for C₃₃H₅₂N₅O₁₄[M+H]⁺ m/z 742.3511, found 742.3799.
tert-butyl 2-(2-(2-(2-(2-(2-(2-(2-(2-(2-aminoethoxy)ethoxy)ethoxy)ethoxy)ethoxy)ethoxy)ethoxy) ethoxy)ethoxy)ethylcarbamate S10 (C₂₅H₂₅N₂O₁₁, 556.69)

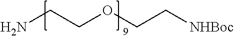

A solution of Boc₂O (0.772 g, 3.54 mmol, 1.01 eg) in CH₂Cl₂ (20 mL) was added dropwise to a solution of S7 (1.6 g, 3.50 mmol) in CH₂Cl₂ (20 mL) over 14 a syringe pump. Upon completion, solvent was removed in vacuo and the residue purified by flash chromatography (CH₂Cl₂/MeOH 1:0 to 8:2) to afford S10 as a colorless oil (0.452 g, 0.812 mmol, 23%). IR (neat): v 3368, 2866, 1708, 1522, 1096 cm⁻¹. ¹H NMR (CDCl₃, 500 MHz): δ5.06 (br s, 1H), 3.66-3.58 (m, 32H), 3.51 (t, J=4.8 Hz, 4H), 3.31-3.26 (m, 2H), 2.88-2.84 (m, 2H), 2.04 (br s, 2H), 1.42 (s, 9H). ¹³C NMR (CDCl₃, 125 MHz): δ 155.9, 79.1, 72.9, 70.7, 70.6, 70.5, 70.4, 70.2, 49.2, 41.7, 40.3, 28.4. HRMS (ESI): calcd for C₂₅H₅₃N₂O₁₁ [M+H]⁺ m/z 557.3649, found 557.3624.
tert-butyl 2-(2-(2-(2-(2-(2-(2-(2-(2-(4′-(2,2,2-trifluoroacetamido)-N-2-benzamidoethoxy)ethoxy)ethoxy)ethoxy)ethoxy)ethoxy)ethoxy) ethoxy)ethoxy)ethylcarbamate S11 (C₃₄H₅₆F₃N₃O₁₃, 771.82)

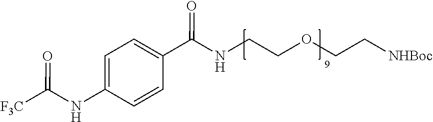

Amide S11 was prepared from S10 (0.452 g, 0.812 mmol) according to the procedure previously reported for S9. The crude material was advanced to the next step without further purification.
tert-butyl 2-(2-(2-(2-(2-(2-(2-(2-(2-(2-benzamidoethoxy)ethoxy)ethoxy)ethoxy)ethoxy)ethoxy) ethoxy)ethoxy)ethoxy)ethylcarbamate 5 (C₃₂H₅₇N₃O₁₂, 675.82)

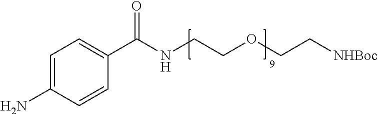

Amide 5 (0.450 g, 0.666 mmol, 82% over 2 steps) was prepared from S11 according to the procedure previously reported for 4. IR (neat): v 3354, 2873, 1699, 1629, 1093 cm⁻¹. ¹H NMR (CDCl₃, 500 MHz): δ 7.63 (d, J=8.6 Hz, 2H), 6.76 (br s, 1H), 6.68 (d, J=8.6 Hz, 2H), 5.07 (br s, 1H), 3.65-3.58 (m, 36H), 3.51 (t, J=5.1 Hz, 2H), 3.31-3.24 (m, 2H), 1.42 (s, 9H). ¹³C NMR (CDCl₃, 125 MHz): δ 167.5, 156.2, 149.3, 128.8, 128.7, 124.0, 114.7, 114.4, 79.2, 70.5, 70.2, 70.1, 69.9, 69.8, 69.7, 47.3, 40.2, 39.4, 28.4. HRMS (ESI): calcd for C₃₂H₅₈N₃O₁₂ [M+H]⁺ m/z 676.4020, found 676.4035.

II b. Synthesis of ARM-F candidates 6-8 and compound 9

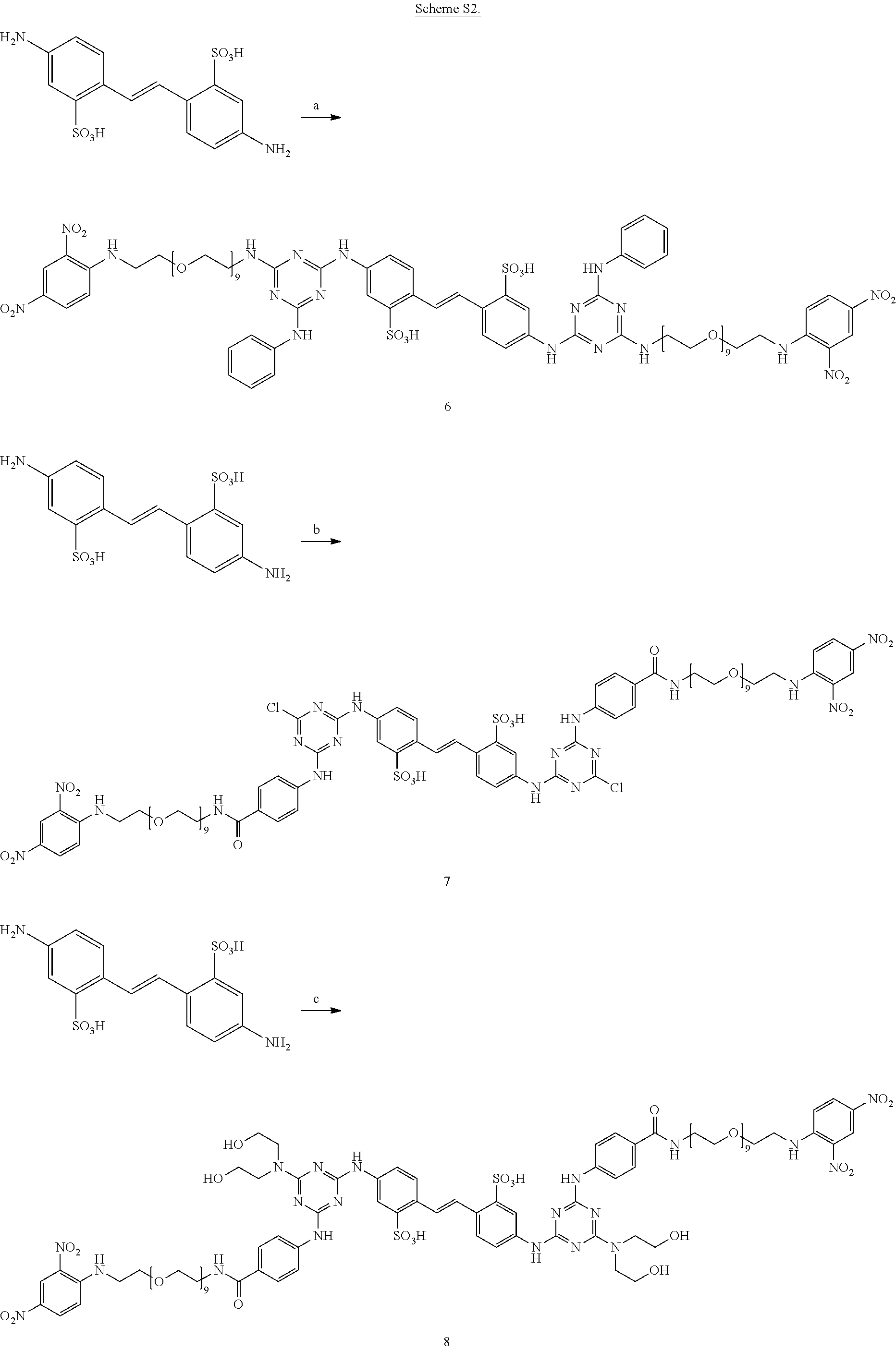

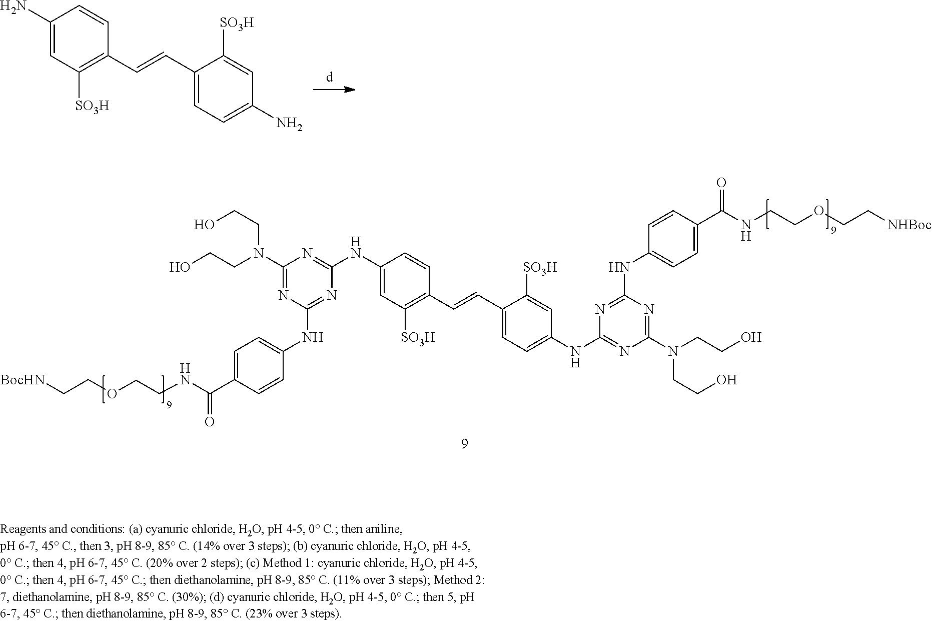

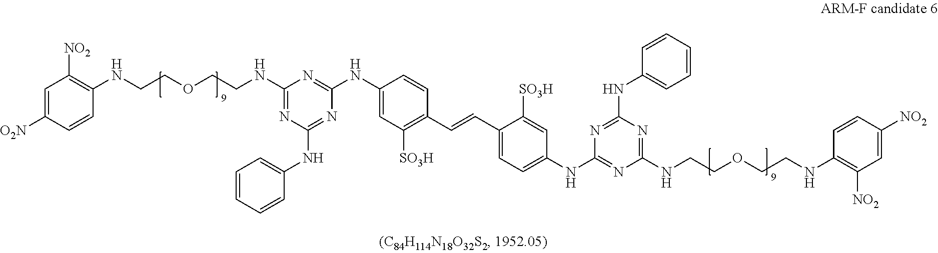

Cyanotic chloride (0.029 g, 0.158 mmol, 2 equiv) was added in small portions to a suspension of 4,4′-dinitrostilbene-2,2′-disulfonic acid (0.030 mg, 0.079 mmol) in water (5 mL) at 0° C. The mixture was stirred at this temperature for 2 h while pH of the mixture was maintained by 4-5 by careful addition of 1M aq. NaOH. Next, pH of the mixture was adjusted to 6-7 by addition of 1M aq. NaOH and aniline (0.015 g, 15 μL, 0.158 mmol, 2 equiv) was added in a single portion. The mixture was stirred for 4 h at 45° C. while pH of the mixture was maintained by 6-7 by addition of 1M aq. NaOH. Finally, 1M aq. NaOH was added to pH 8-9 followed by addition of a solution of 3 (0.97 g, 0.316 mmol, 4 equiv) in MEK (1 mL). The mixture was ,stirred for another 4 h at 85° C. while pH of the mixture was maintained by 8-9 by addition of 1M aq. NaOH. Then, the mixture was cooled to 0° C., acidified to pH 2 by addition of 1M aq HCl and the redorange precipitate was collected by filtration. Purification by prep. HPLC (0.15M aq. NH₄HCO₃/MeCN 100-0 to 0-100 over 36 min) afforded 6 as a yellow powder (0.022 g, 0.011 mmol, 14% over 3 steps). IR (neat): v 3351, 2918, 1619, 1490, 1417 cm⁻¹. ¹H NMR ((CD₃)₂SO, 600 MHz): δ 9.12 and rotamers 9.26, 8.99 (br s, 2H), 8.85 (s, 2H), 8.25 (d, J=8.9 Hz, 2H), 8.04-7.98 (m, 2H), 7.87-7.76 (m, 4H), 7.62-7.53 (m, 2H), 7.29-7.21 (m, 4H), 6.93 (s, 2H), 6.56 (s, 1H). ¹³C NMR ((CD₃)₂SO, 150 MHz): δ 166.1, 164.5, 148.8, 135.3, 130.3, 130.1, 128.7, 124.0, 120.0, 116.1, 70.2, 70.1, 69.5, 68.6, 43.1. HRMS (ESI): calcd for C_{S4}H₁₂₂N₂₀O₃₂S₂ [M+2NH₄]²⁺ m/z 993.3987, found 993.3958.

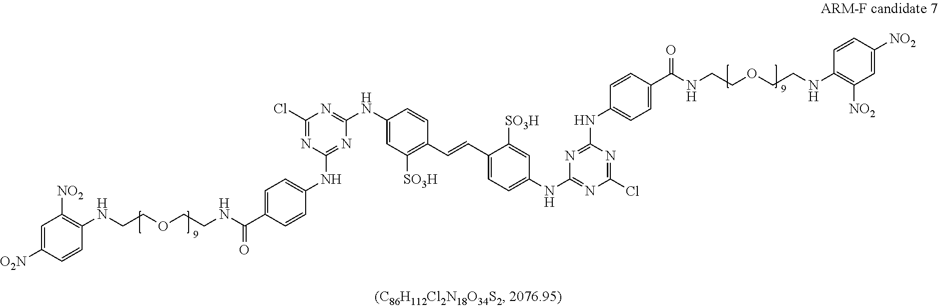

Cyanuric chloride (0.037 g, 0.200 mmol, 2 equiv) was added in small portions to a suspension of 4,4′-dinitrostilbene-2,2′-disulfonic acid (0.037 g, 0.100 mmol) in water (7.5 mL) at 0° C. The mixture was stirred at this temperature for 2 h while pH of the mixture was maintained by 4-5 by careful addition of 1M aq. NaOH. Next, pH of the mixture was adjusted to 6-7 by addition of 1M aq. NaOH and a solution of 4 (0.148 g, 0.200 mmol, 2 equiv) in MEK (1 mL) was added in a single portion. The mixture was stirred for 4 h at 45° C. while pH of the mixture was maintained by 6-7 by addition of 1M aq. NaOH. Upon completion, the mixture was cooled to 0° C., acidified to pH 2 by addition of 1M aq HCl and the red-orange precipitate was collected by filtration. Purification by prep. HPLC (0.15M aq. NH₄HCO₃/MeCN 100-0 to 0-100 over 36 min) afforded 7 as a yellow powder (0.041 g, 0.020 mmol, 20% over 2 steps). IR(neat): v 3357, 2917, 1619, 1487, 1304 cm⁻¹. ¹H NMR ((CD₃)₂SO, 600 MHz): δ 9.59 (br s, 2H), 8.85 (s, 2H), 8.39-8.34 (m, 2H), 8.24 (dd, J₁=9.6 Hz, J₂=2.6 Hz, 2H), 8.13-8.09 (m, 1H), 8.02-7.89 (m, 3H), 7.87-7.78 (m, 5H), 7.71 (d, J=8.9 Hz, 1H), 7.26 (d, J=9.7 Hz, 2H), 3.70-3.61 (m, 8H), 3.61-3.41 (m, 72H). ¹³C NMR ((CD₃)₂SO, 150 MHz): δ 166.3, 164.3, 148.8, 145.9, 143.2, 135.3, 130.3, 130.0, 128.2, 127.7, 124.0, 119.2, 116.1, 70.2, 70.1, 70.0, 69.4, 68.7, 43.1. HRMS (ESI): calcd for C_{S6}H₁₂₀Cl₂N₂₀O₃₄S₂ [M+2NH₄]²⁺ m/z 1055.3547, found 1055.3582.

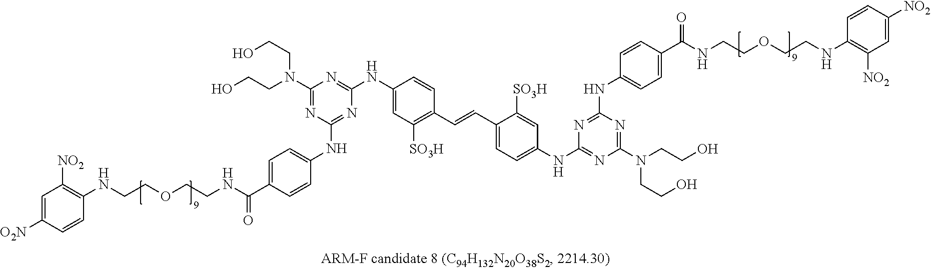

Method 1 (“one-pot” synthesis). Cyanuric chloride (0.055 g, 0.300 mmol, 2 equiv) was added in small portions to a suspension of 4,4′-dinitrostilbene-2,2′-disulfonic acid (0.055 g, 0.150 mmol) in water (12 mL) at 0° C. The mixture was stirred at this temperature for 2 h while pH of the mixture was maintained by 4-5 by careful addition of 1M aq. NaOH. Then, pH of the mixture was adjusted to 6-7 by addition of 1M aq. NaOH and a solution of 4 (0.222 g, 0.300 mmol, 2 equiv) in MEK (1.5 mL) was added in a single portion. The mixture was stirred for 4 h at 45° C. while pH of the mixture was maintained by 6-7 by addition of 1M aq. NaOH. Next, 1M aq. NaOH was added to pH 8-9 Followed by addition of diethanolamine (165 μL, 0.180 g, 1.20 mmol, 8 equiv). The mixture was stirred for another 4 h at 85° C. while pH of the mixture was maintained by 8-9 by addition of 1M aq. NaOH. Then, the mixture was cooled to 0° C., acidified to pH 2 by addition of 1M aq HCl and the red-orange precipitate was collected by filtration. Purification by prep. HPLC (0.15M aq. NH₄HCO₃/MeCN 100-0 to 0-100 over 36 min) afforded 8 as a bright yellow powder (0.036 g, 0.016 mmol, 11% over 3 steps).

Method 2 (from 7). Diethanolamine (35 μL, 0.036 g, 0.346 mmol, 8 equiv) was added to a suspension of 7 (0.090 g, 0.043 mmol) in H₂O (10 mL) and MEK (1 mL) and the mixture was heated to reflux for 3 h while pH of the mixture was maintained by 6-7 by addition of 1M aq. NaOH. Upon completion, the mixture was cooled to 0° C., acidified to pH 2 by addition of 1M aq HCl and the red-orange precipitate was collected by filtration. Purification by prep. HPLC (0.15M aq. NH₄HCO₃/MeCN 100-0 to 0-100 over 36 min) afforded 8 as a bright yellow powder (0.028 g, 0.013 mmol, 30% from 7). IR (neat): v 3347, 2921, 1619, 1537, 1411 cm⁻¹. ¹H NMR ((CD₃)₂SO, 600 MHz): δ 9.31 (br s, 2H), 8.85 (s, 2H), 8.36-8.30 (m, 2H), 8.25 (d, J=8.9 Hz, 2H), 8.01 (s, 2H), 7.94-7.88 (m, 4H), 7.76 (d, J=8.1 Hz, 4H), 7.59-7.54 (m, 2H), 7.28 (d, J=9.2 Hz, 2H), 6.65 (s, 1H), 4.91 (br s, 2H), 4.78 (s, 2H), 3.79-3.61 (m, 24H), 3.58-3.42 (m, 76H), 3.42-3.36 (m, 4H, overlapping with H₂O signal). ¹³C NMR ((CD₃)₂SO, 150 MHz): δ 166.4, 165.3, 164.1, 148.8, 145.7, 143.7, 135.3, 130.3, 130.1, 128.1, 127.2, 125.7, 124.0, 118.7, 116.1, 70.2, 70.1, 70.0, 69.4, 68.7, 59.5, 50.9, 43.1. HRMS (ESI): calcd for C₉₄H₁₄₀N₂₂O₃₈S₂ [M+2NH₄]²⁺ m/z 1124.4570, found 1124.4472.

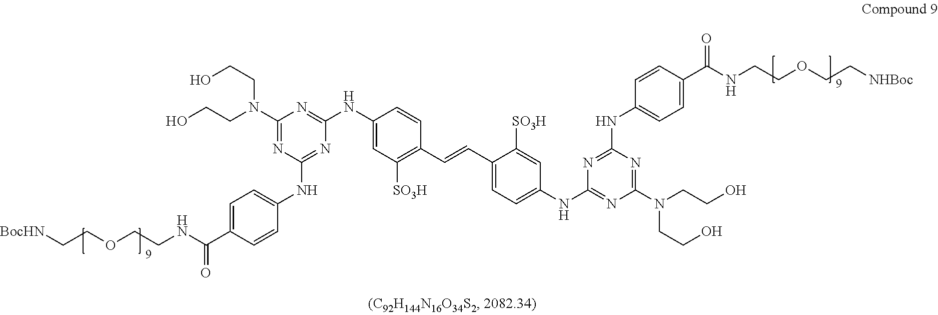

Compound 9 was prepared according to the procedure previously reported for 8 (method 1). Purification by prep. HPLC (0.15M aq. NH₄HCO₃/MeCN 100-0 to 0-100 over 36 min) afforded 9 as an off-white powder (0.043 g, 0.021 mmol, 23% over 3 steps). IR (neat): v 3304, 2921, 1613, 1482, 1407 cm⁻¹. ¹H NMR ((CD₃)₂SO, 600 MHz): δ 9.32 (br s, 2H), 8.37-8.31 (m, 2H), 8.00 (s, 2H), 7.94-7.88 (m, 2H), 7.77 (d, J=8.4 Hz, 4H), 7.60-7.54 (m, 2H), 6.75 (s, 2H), 6.57 (s, 1H), 4.92 (br s, 2H), 4.78 (br s, 2H), 3.78-3.59 (m, 18H), 3.56-3.44 (m, 74H), 3.43-3.38 (m, 4H), 3.07-3.02 (m, 4H), 1.36 (s, 18H). ¹³C NMR ((CD₃)₂SO, 150 MHz): δ 166.4, 165.3, 164.1, 156.0, 145.8, 128.1, 127.2, 125.7, 118.7, 78.0, 70.2, 70.1, 70.0, 69.6, 69.5, 59.5, 50.9, 28.7. HRMS (ESI): calcd for C₉₂H₁₅₂N₁₈O₃₄S₂ [M+2NH₄]²⁺ m/z 1058.5080, found 1058.4985.

### Biological Evaluation

I. ELISA Assay for Antibody Recruitment

Free chitin (0.100 g, Alfa Aesar, United Kingdom) was hydrolyzed by adding 10 ml of conc. HCl for 30 min with gentle stirring. An aliquot of the chilin solution(1 mL) was mixed with 9 ml of PBS to the final concentration of 1 mg/mL. This solution was further diluted to 100 μg/mL with PBS and coated overnight at 4° C. in a 96-well Nunc MaxiSorp microtiter plate. Upon completion, the coated plate was blocked with 2% BSA solution at 37° C. for 3 h. Next, compounds and controls at predetermined dilutions made in the 2% BSA blocking solution were added followed by addition of 2 mg/ml anti-DNP biotinylated antibodies diluted 1:100 (Invitrogen, CA). The plate was covered and incubated at 37° C. for 1 h. The plate was washed 3 times with wash solution (0.2% Tween 20 in PBS) and HRP-labeled streptavidin secondary reagent was added at a dilution 1:10000 in 2% BSA. The plate was incubated again at 37° C. for 1 h followed by washing 3 times with wash solution. HRP substrate TMB Ultra solution (Thermo Fischer, IL) was added to the plates and incubated until suitable color development and the reaction was stopped by adding 100 μL of aq. 1N H₂SO₄. Quantification was performed by measuring absorbance on a plate reader at 450 nm.

II. Fungal Cell Strains and Culture

Cultures of C. albicans Ca2323 were grown in Sabourad dextrose broth (Hardy Diagnostics, CA) to maintain them in a non-sporulating yeast form (blastopore form). S. cerevisiae YPH499 (Mat type a) were grown in YPD brath (Teknova, CA). Chitin-deficient strain S. cerevisiae YMS348s Δchs 1,2,3 was grown in YPD broth supplemented with 1M sorbitol. The fungal cultures were generally grown overnight at 30° C. for the antibody recruitment and phagocytosis assays. For consistency, O.D. measurements were taken using a single cuvette UltraSpec 10 cell density meter and experiments were performed on cultures with similar density. For experiments involving caspofungin treated C. albicans, the cells were grown overnight in media containing 1.25 ng/ml caspofungin acetate (Merck, PA). For experiments involving α factor induced arrest, α factor pheromone trifluoroacetate salt (Bachem, Switzerland) was added to a fresh S. cerevisiae YPH499 culture to a final concentration of 5 μg/mL followed by incubation for 1 h. Then, the cells were treated repeatedly with α factor pheromone trifluoroacetate salt (5 μg/mL) and incubated for an additional 2 h.

III. Antibody Recruitment Assay

Fungal cells for the antibody recruitment experiments were always grown to 1.0 O.D. for consistency across experiments. The fungal cells were pelleted, washed, counted on the flow cytometer and re-constituted in FACS buffer of PBS containing 5% BSA at a density of 1×10⁵ cells/mL. One microliter of suitably diluted ARM compound followed by 1 μL of 1:100 diluted anti-DNP biotinylated mouse monoclonal antibody (2 mg/mL, Invitrogen, CA) was added to 100 μL of fungal cells in FACS buffer. The reaction was mixed well and incubated in a 37° C. incubator for 30 minutes. Post incubation, washing was performed by adding 1 mL of FACS buffer to the reaction and the cells pelleted by centrifugation at 10000x g for 5 minutes. The pelleted cells were then reconstituted in 100 μl of FACS buffer followed by addition of 1 μL of 1:1000 diluted AF647 labeled streptavidin (0.5 mg/mL, BioLegend, CA) and 1 μL of 100 μg/ml propidium iodide (PromoKine, Germany). The cells were incubated for a further 30 min and the wash procedure was repeated. All experiments consisted of two sets of controls: (i) flow controls: unstained cells, propidium iodide stained cells, secondary reagent stained cells; (ii) background controls: compound 9 lacking DNP motifs, and no ARM-F. The cells were analyzed by flow cytometry on an Attune Nxt flow cytometer (Thermo Fischer Scientific) and the raw data further analyzed using FlowJo software (FlowJo LLC, OR).

IV. Effector Cell Line Culture

The pro-myeloblast cell line HL-60 was obtained from AtCC and cultured in high serum DMEM-F12 growth medium containing 15% FBS, 1 mM sodium pyruvate, 2 mM L-glutamine, 1X non-essential amino acids with antibiotics penicillin (10 U/mL) and streptomycin (10 μg/mL). The cells were maintained at low passage at 37° C., 5% CO₂ throughout the experiments. The cells were counted every 24-48 hours on a Roche Innovatis Cedex XS automated cell counter and generally maintained at a density of 1×10⁵ cells/mL. When necessary, neutrophil differentiation was achieved by adding DMF (final concentration 0.08%) to the culture media containing required number of cells and incubating at 37° C., 5% CO₂ for greater than 24 hours.

V. Dual Color Flow Cytometry-Based Fungal Opsono-Phagocytosis Assay

Fungal cells for these experiments were always grown to 1.0 OD and the HL-60 cells to approximately 1×10⁵ cells/mL for consistency between experiments. The fungal cells were counted by flow cytometry, pelleted and reconstituted in a fixing solution of ice-cold 70% ethanol and incubated for 1 h. The fixed cells were pelleted and washed five times by adding 10 mL of PBS followed by pelleting. After the final wash, the cells were re-suspended in FITC staining solution (750 μg/mL FITC in PBS) and incubated at 37° C. for 45 mm. The cells were pelleted and washed 5 times by adding 10 mL of PBS followed by pelleting. After the final wash, the fungal cells were counted again on the flow cytometer and re-constituted in FACS buffer of PBS containing 5% BSA at a density of 2×10⁶ cells/mL. Neutrophil differentiated HL-60 cells were counted on the Roche Inaovatis Cedex XS automated cell counter with inclusion of trypan blue staining (0.1%) for assessment of viability. Phagocytosis experiments were performed with cell cultures exhibiting >80% viability.

For each phagocytosis reaction 1.0×10⁵ fungal cells were suspended in a 50 μL volume of HL-60 growth media. One microliter of appropriately diluted compound (stock solutions in DMSO) was loaded and mixed followed by addition of 1 μL 1:00 dilution of rabbit anti-DNP polyclonal antibodies (2 mg/mL, Life Technologies). This was followed by addition of 50 μL (1.0×10⁵) activated HL-60 cells and incubated at 37° C. for 1 h. After completion of the phagocytosis incubation period, 1 μL of 1:10 dilution of anti-Candida polyclonal rabbit antibodies (4 mg/mL) were added followed by addition of 1 μL of 1:20 dilution Of AF647 labeled anti-rabbit secondary antibodies (2 mg/mL, Life Technologies, CA) and incubated for further 5 min at 37° C. The HL-60 cells were disrupted by osmotic shock by adding 1 mL of de-ionized water. The mix was vortexed and flow cytometry analysis performed on the Attune Nxt flow cytometer (Thermo Fischer Scientific). The forward and side scatter gates and the fluorescence signal quadrant gates were set using FITC stained fungal cells also stained with the anti-Candida AF 647 stain. In the phagocytosis reactions, the FITC, AF647 double positive cells were considered to be representative of non-phagocytosed cells and FITC single positive cells representative of phagocytosed cells protected in the HL-60 intracellular environment and released upon their osmotic destruction. Total phagocytosis was calculated as a percentage of FITC single positive cells out of all stained fungal cells (single or double positive).

1. A compound according to the chemical structure: 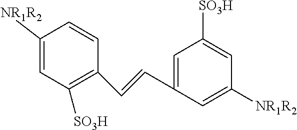 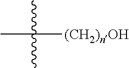
Wherein each R₁ is independently H, C₁-C₃ alkyl or a group; 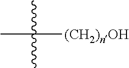 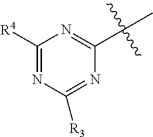
Each R₂ is H, a group or a group, with the proviso that at 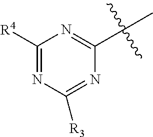
least one R₂ group is a group;
Each n′ is independently 1-6; 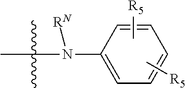
Each R₃ is independently H, Cl or a group;
Where R^{N} is H or a C₁-C₃ alkyl group optionally substituted with one or two hydroxyl groups;
Each R₅ is independently H, CO₂R^{E}, SO₃H, L-P_{G} or L-ABT;
R^{E} is H, a C₁-C₆ alkyl group or a L-ABT group;
P_{G} is a protecting group; 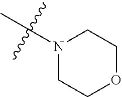 [SMILES: *N1CCOCC1] 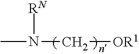
Each R⁴ is independently H, Cl, a L-ABT group or a 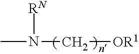
group with the proviso that at least one R⁴ is a L-ABT group or a group;
Where R^{N} and n′ are the same as above; and
R¹ is H, P_{G} or a L-ABT group;
Where L is a linker group optionally containing at least one connector group CT; and
ABT is an antibody binding moiety comprising a hapten which is capable of binding to an antibody present in a patient prior to the administration of the compound to the patient, or a pharmaceutically acceptable salt, stereoisomer, enantiomer, solvate or polymorph thereof.

2. The compound according to claim 1 wherein R₁ and R^{N} are each H.

3. The compound according to claim 1 wherein each R₂ group is

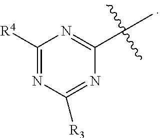

4. The compound according to claim 1 wherein R₃ is a 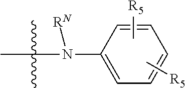
group where R₅ is H, SO₃H, CO₂R^{E} or L-ABT.

5. The compound according to claim 4 wherein each R₅ group is L-ABT.

6. The compound according to claim 5 wherein said R₄ groups are substituted on the phenyl group at the ortho and para position (positions 2 and 4 of the phenyl group).

7. The compound according to claim 5 wherein said R₄ groups are substituted on the phenyl group at the ortho and meta positions.

8. The compound according to claim 7 wherein said R₄ groups are substituted on the phenyl group at positions 2 and 4 of the phenyl ring.

9. The compound according to claim 1 wherein 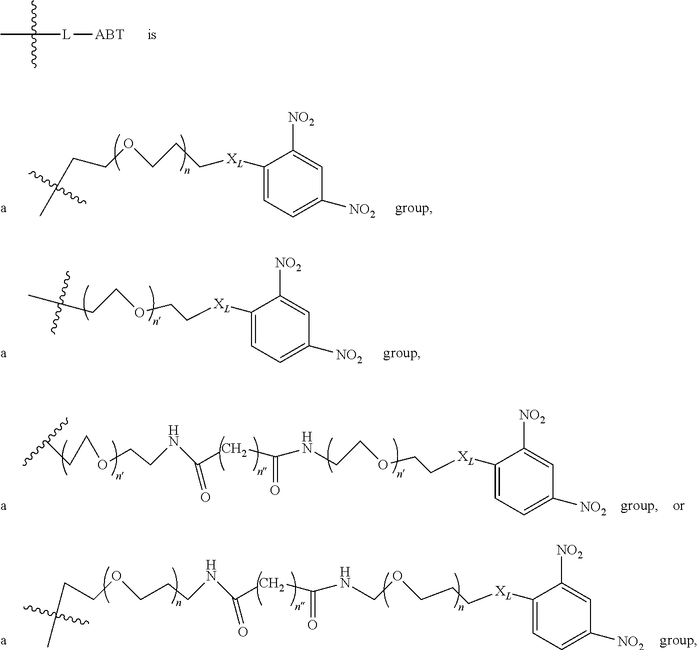
where X_{L} is N(R¹), O, S, S(O), SO₂, S(O)₂O, —OS(O)₂, or OS(O)₂O; and
R₁ is H, a C₁-C₃ alkyl group or a —C(O)(C₁-C₃) group, preferably H;
each n and n′ is independently 1 to 25, 1 to 15, 1 to 12, 2 to 11, 2 to 10, 2 to 8, 2 to 6, 2 to 5, 2 to 4 and 2 to 3 or 1, 2, 3, 4, 5, 6, 7, or 8; and
each n″ is independently 0 to 8, often 1 to 7, or 1, 2, 3, 4, 5 or 6 (preferably 3).

10. The compound according to claim 1 wherein L is a linker group based upon polyethyleneglycol (PEG) linkages, polypropylene glycol linkages, or polyethyleneglycol-co-polypropylene oligomers of up to 100 ethyhlene glycole or propylene glycol units (about 1 to 75, about 1 to 60, about 1 to 50, about 1 to 35, about 1 to 25, about 1 to 20, about 1 to 15, 2 to 10, about 4 to 12, about 1 to 8, 1 to 3, 1 to 4, 2 to 6, 1 to 5, etc.).

11. The compound according to claim 1 wherein L is a linker group according to the chemical structure: 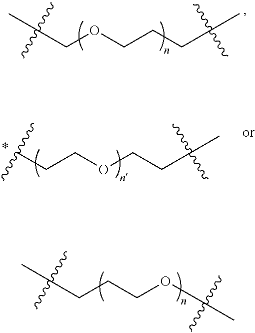
where each n and n′ is independently 1 to 25, 1 to 15, 1 to 12, 2 to 11, 2 to 10, 2 to 8, 2 to 6, 2 to 5, 2 to 4 and 2 to 3 or 1, 2, 3, 4, 5, 6, 7, or 8.

12. The compound according to claim 1 wherein L is a linker group according to the chemical structure: 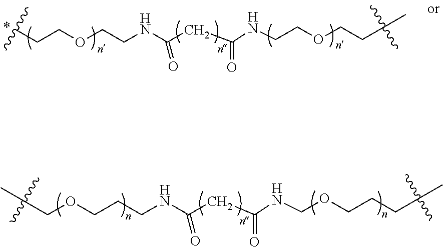
where each n and n′ is independently 1 to 25, 1 to 15, 1 to 12, 2 to 11, 2 to 10, 2 to 8, 2 to 6, 2 to 5, 2 to 4 and 2 to 3 or 1, 2, 3, 4, 5, 6, 7, or 8; and
each n″ is independently 0 to 8, often 1 to 7, or 1, 2, 3, 4, 5 or 6.

13. The compound according to claim 1 wherein L is a polyamino acid optionally comprising one or two connector groups CT comprising up to 100 (preferably about 1 to 75, about 1 to 60, about 1 to 50, about 1 to 45, about 1 to 35, about 1 to 25, about 1 to 20, about 1 to 15, 2 to 10, about 4 to 12, about 5 to 10, about 4 to 6, about 1 to 8, about 1 to 6, about 1 to 5, about 1 to 4, about 1 to 3) amino acid residues wherein said amino acid residues are selected from naturally occurring D and L amino acids or L is a group according to the chemical structure: 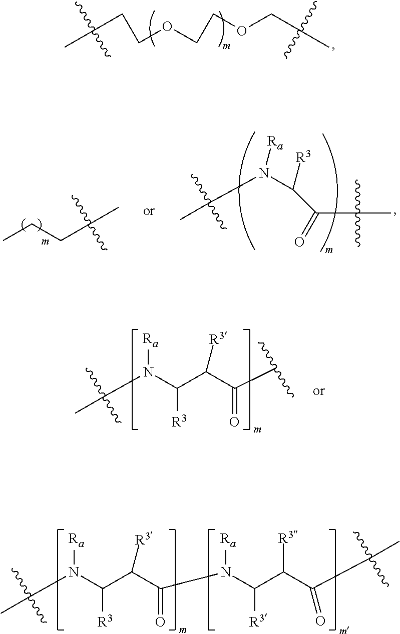
Where Rₐ and R_{a′} are each independently H, C₁-C₃ alkyl, alkanol, aryl or benzyl
or each forms a cyclic ring with R³ or R^{3′} on an adjacent carbon respectively (to form proline or hydroxyproline) or R³, R^{3′} and R^{3″} are each independently a side chain derived from an amino acid preferably selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline, hydroxylproline (R³ or R^{3′} forms a cyclic ring with Rₐ or R_{a′} and the adjacent nitrogen group to form a pyrrolidine group for proline or a hydroxypyrrolidine for hydroxyproline), serine (methanol), threonine (ethanol, 1-hydroxyethane), tryptophan (methyleneindole), tyrosine (methylene phenol) or valine (isopropyl); and m and m′ (within the context of this use) is each independently an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5.

14. The compound according to claim 1 wherein L is a linker group according to the chemical formula: 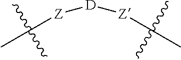
Where Z and Z′ are each independently a bond, —(CH₂)ᵢ—O, —(CH₂)ᵢ—S, —(CH₂)ᵢ—N—R, 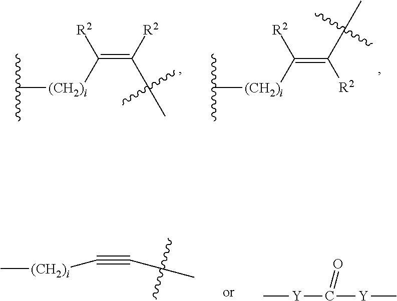
wherein said —(CH₂)ᵢ group, if present in Z or Z′, is bonded to a connector (CT), an alternative linker, A_{B}M and/or UPAR_{B}M;
Each R is H, or a C₁-C₃ alkyl or alkanol group;
Each R² is independently H or a C₁-C₃ alkyl group;
Each Y is independently a bond, O, S or N—R;
Each i is independently 0 to 100, 0 to 75, 1 to 60, 1 to 55, 1 to 50, 1 to 45, 1 to 40, 2 to 35, 3 to 30, 1 to 15, 1 to 10, 1 to 8, 1 to 6, 0, 1, 2, 3, 4 or 5;
D is 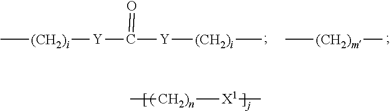
or
a bond, with the proviso that Z, Z′ and D are not each simultaneously bonds;
j is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
m′ is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
n is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5 (n is preferably 2);
X¹ is O, S or N—R; and
R is as described above,
or a pharmaceutical salt thereof.

15. A compound according to claim 1 wherein linker group L is a group according to the chemical structure: 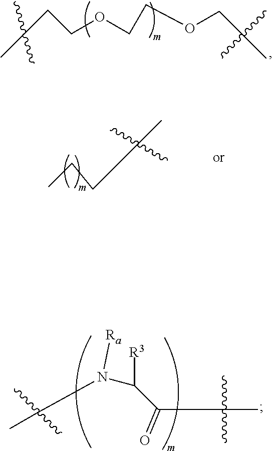
or
a polypropylene glycol or polypropylene-co-polyethylene glycol linker containing between 1 and 100 alkyleneglycol units;
Where Rₐ is H, C₁-C₃ alkyl or alkanol or forms a cyclic ring with R³ when R³ is a sidechain of proline or R³ is a side chain derived from a naturally occurring D- or L-amino acid; and
Each m is independently an integer from 1 to 100.

16. A compound according to claim 1 wherein said ABT group is a group according to the chemical structure: 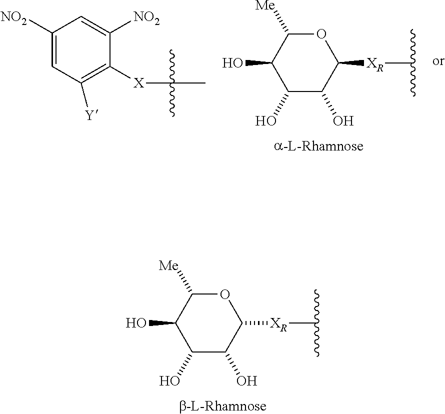
Where Y′ is H or NO₂ (preferably H);
X is O, CH₂, NR¹, S(O), S(O)₂, —S(O)₂O, —OS(O)₂, or OS(O)₂O; and
R¹ is H, a C₁-C₃ alkyl group, or a —C(O)(C₁-C₃) group;
and
X_{R} is O, S or NR¹.

17. The compound according to claim 1 wherein said ABT group is a group according to the chemical structure: 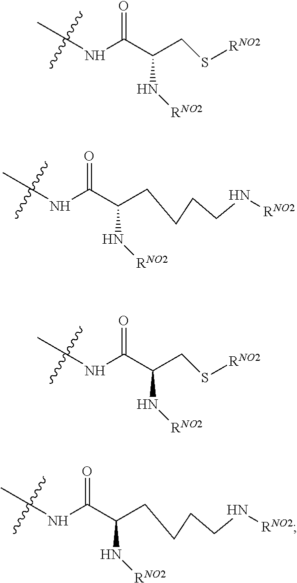
Where R^{NO2} is a nitrophenyl or a dinitrophenyl group linked through an amino or thiol group as indicated;
or a group according to the chemical structure: 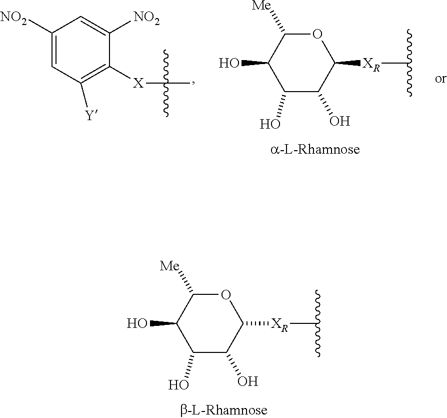
Where Y′ is H;
X is O, CH₂, NR¹, S(O), S(O)₂, —S(O)₂O, —OS(O)₂, or OS(O)₂O; and
R¹ is H, a C₁-C₃ alkyl group, or a —C(O)(C₁-C₃) group.

18. The compound according to claim 1 wherein said ABT group
is a group represented by the chemical formula: 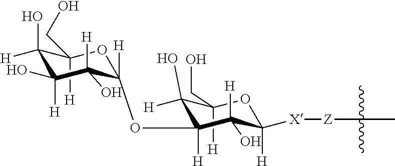
Where X′ is CH₂, O, N—R¹′, or S, preferably O;
R^{1′} is H or C₁-C₃ alkyl; and
Z is a bond, a monosaccharide, disaccharide, oligosaccharide, glycoprotein or glycolipid.

19. The compound according to claim 1 wherein said ABT group is a group according to the chemical structure: 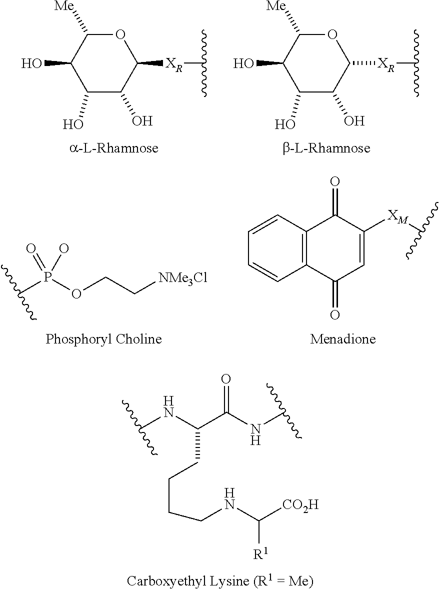
Where X_{R} is O, S or NR¹; and
X_{M} is O, NR¹ or S, and
R¹ is H or a C₁-C₃ alkyl group.

20. The compound according to claim 15 wherein said ABT group comprises from one to four rhamnose groups.

21. The compound according to claim 1 wherein said ABT group is a group according to the chemical structure: 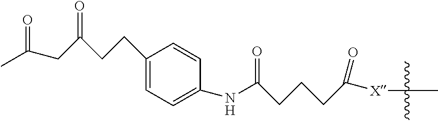
Where X″ is O, CH₂, NR¹, S; and
R¹ is H, a C₁-C₃ alkyl group or a —C(O)(C₁-C₃) group; or 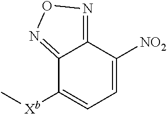
a group;
Where X^{b} is a bond, O, CH₂ or NR¹ or S; and
R¹ is the same as above; or
a group according to the chemical structure: 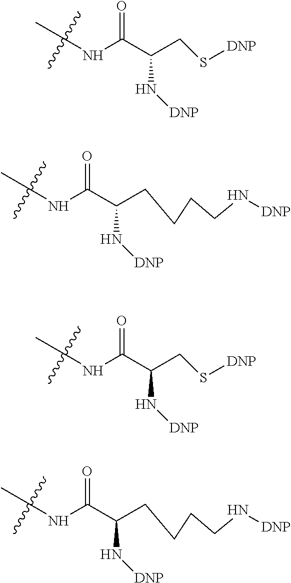
Where DNP is a dinitrophenyl group; or
a dinitrophenyl group according to the chemical structure: 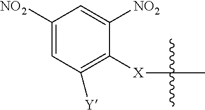
Where Y′ is H;
X is CH₂, C(O), NR¹, S(O), S(O)₂, —S(O)₂O, —OS(O)₂, or OS(O)₂O; and
R¹ is H, a C₁-C₃ alkyl group, or a —C(O)(C₁-C₃) group.

22. The compound according to claim 1 wherein said ABT group is a dinitrophenyl group or a rhamnose group.

23. The compound according to claim 1 wherein said ABT group is a dinitrophenyl group.

24. The compound according to claim 1 wherein said CT group is a group according to the chemical structure: 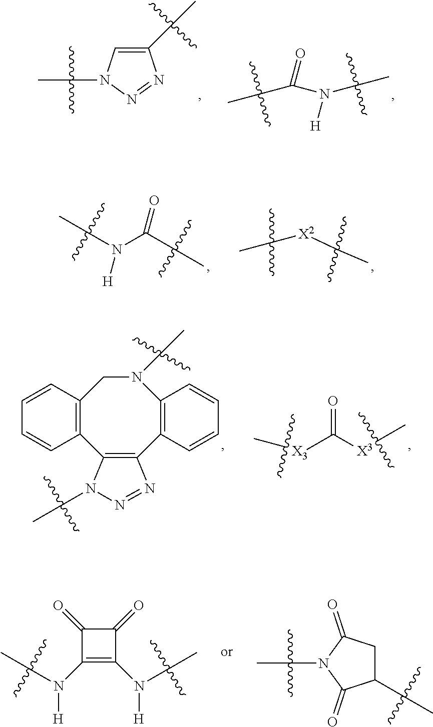
or a diamide group according to the structure: 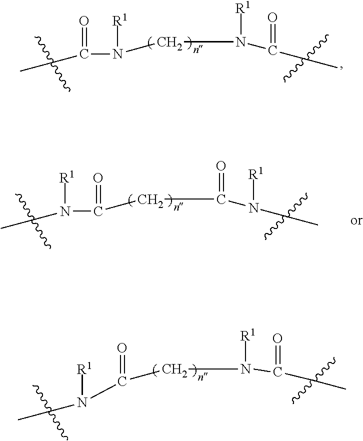
Where X² is CH₂, O, S, NR⁴, C(O), S(O), S(O)₂, —S(O)₂O, —OS(O)₂, or OS(O)₂O;
X³ is O, S, NR⁴;
R⁴ is H, a C₁-C₃ alkyl or alkanol group, or a —C(O)(C₁-C₃) group;
Each R¹ is independently H or a C₁-C₃ alkyl group (preferably H); and
n″ is independently 0 to 8, often 1 to 7, or 1, 2, 3, 4, 5 or 6 (preferably 3).

25. The compound according to claim 23 wherein said CT group is a group according to the chemical structure: 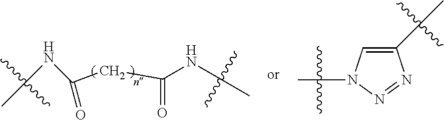
Where n″ is 1-7.

26. A compound according to the chemical structure: 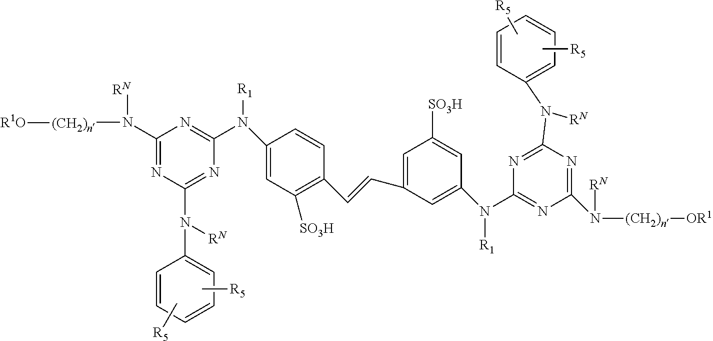
Where each R₁ and R^{N} is independently H or a C₁-C₃ alkyl group which is optionally substituted with one or two hydroxyl groups;
R₅ is independently H, SO₃H, L-P_{G} or L-ABT;
L is a linker group containing at least one connector CT group;
PG is a protecting group;
ABT is an antibody binding moiety comprising a hapten which is capable of binding to an antibody present in a patient;
Each n′ is independently 1-6; and
Each R¹ is a L-Pg group or a L-ABT group, preferably according to the chemical structure: 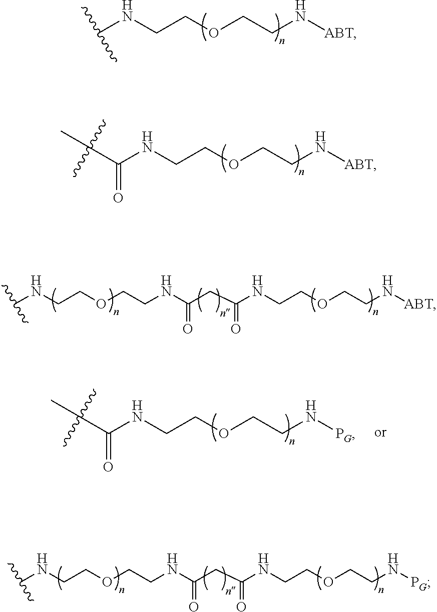
where each n is independently 1-45;
n″ is 1-10;
P_{G} is a protecting group and
ABT is an antibody binding group; or
a pharmaceutically acceptable salt, stereoisomer (a diastereomer or enantiomer), solvate or polymorph thereof.

27. A compound according to the chemical structure: 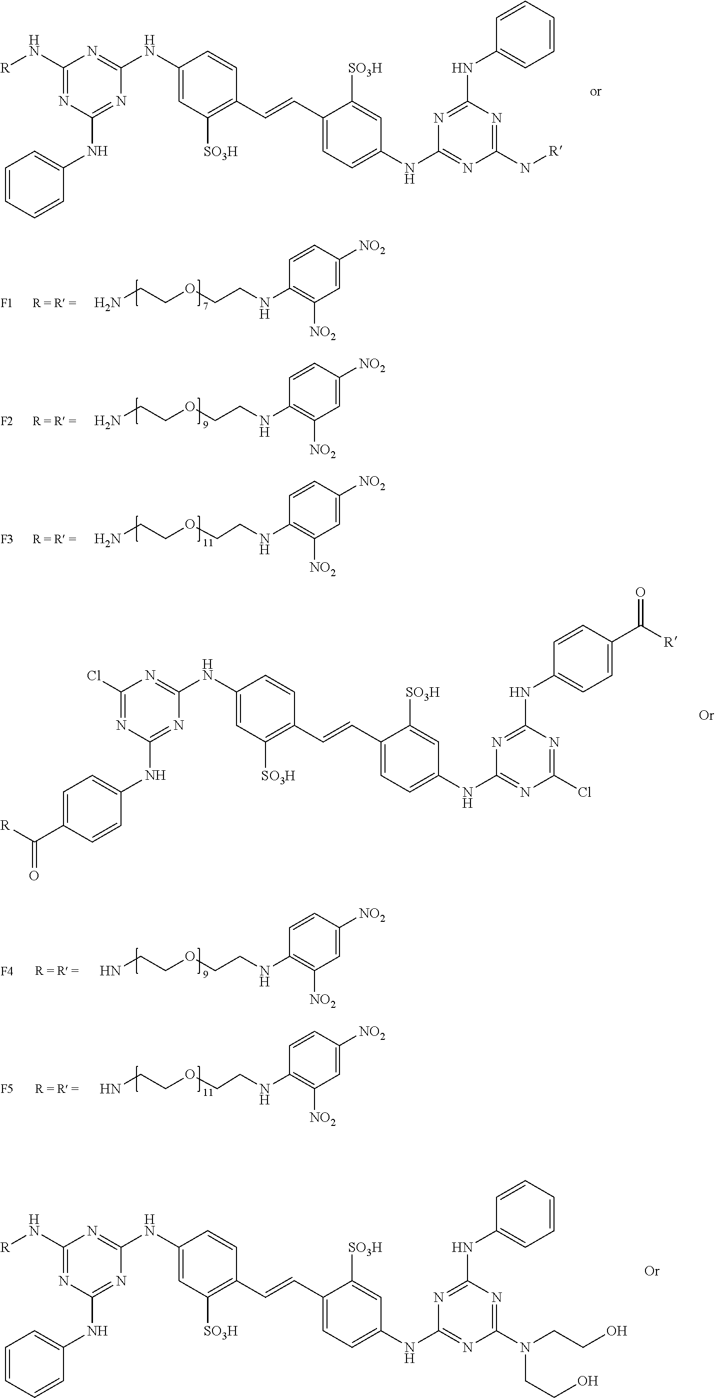 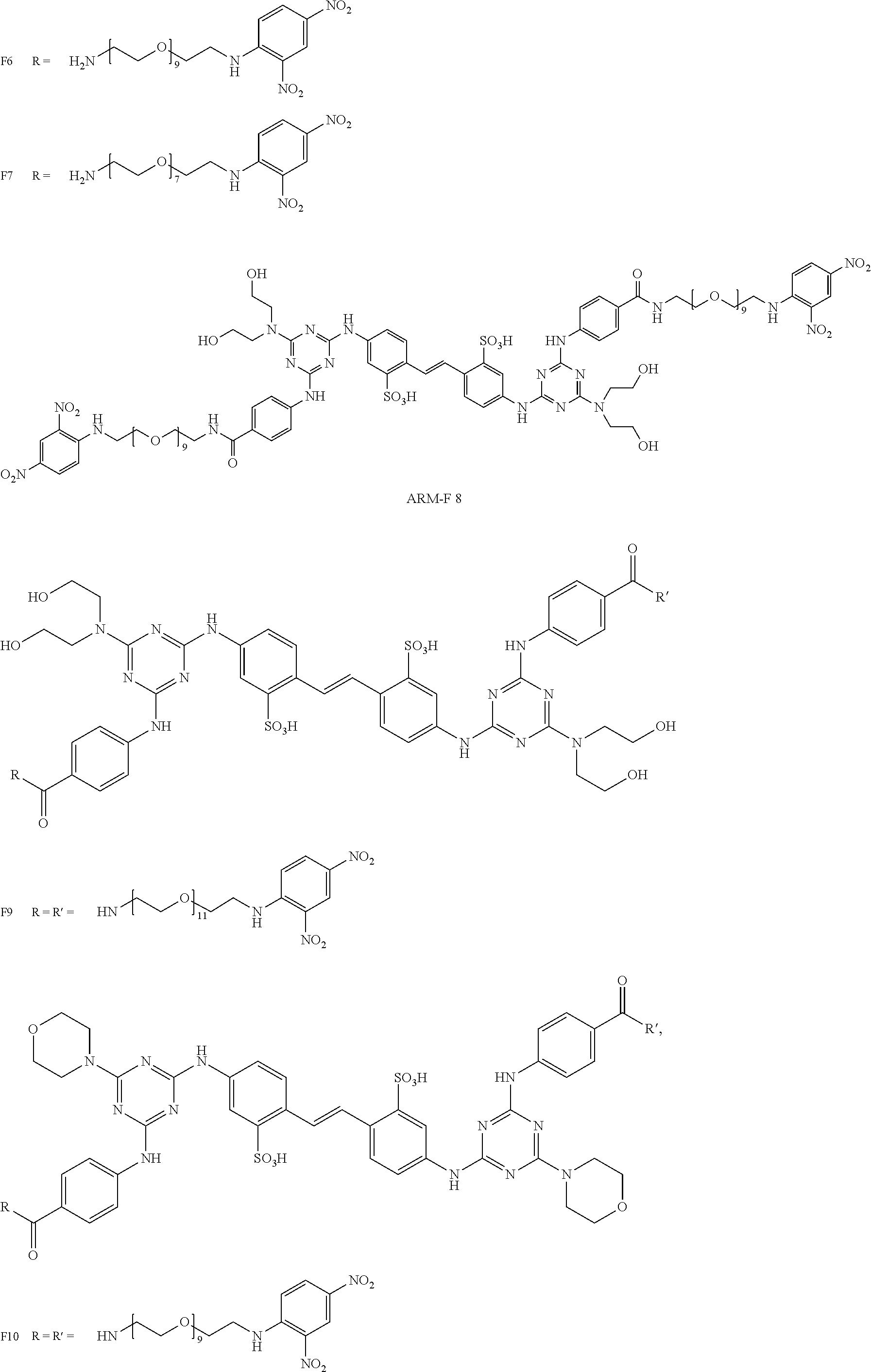
or
A pharmaceutically acceptable salt, stereoisomer, solvate or polymorph thereof.

28. A compound according to FIG. 5, scheme 4 hereof, or a pharmaceutically acceptable salt, stereoisomer, solvate or polymorph thereof.

29. A compound of claim 27 according to the chemical structure: 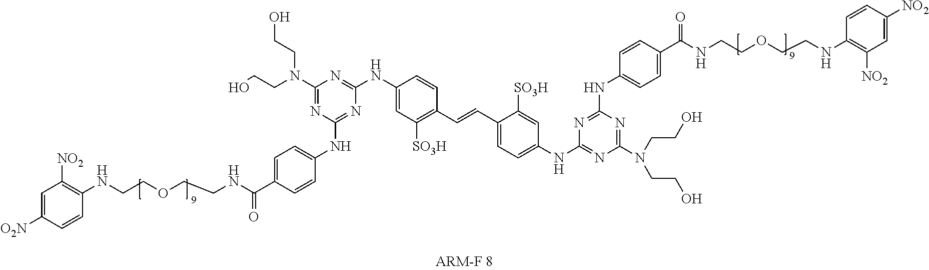
Or a pharmaceutically acceptable salt, stereoisomer, solvate or polymorph thereof.

30. A pharmaceutical composition comprising an effective amount of a compound according to claim 1, in combination with a pharmaceutically acceptable carrier, additive or excipient.

31. A pharmaceutical composition according to claim 30 further in combination with an additional antifungal agent.

32. The composition according to claim 31 wherein said additional antifungal agent is a polyene, imidazole, triazole, allylamine, echinocandin, a miscellaneous antifungal agent or a mixture thereof.

33. The composition according to claim 31 wherein said additional antifungal agent is selected from the group consisting of nystatin, amphotericin B, ketoconazole, clotrimazole, fluconazole, itraconazole, posaconazole, voriconazole, terbinafine, anidulafungin, caspofungin, micafungin, flucytosine, griseofulvin, pentamine and mixtures thereof.

34. The composition according to claim 33 which includes caspofungin.

35. A method of treating a fungal infection in a patient in need comprising administering to said patient an effective amount of a compound according to claim 1.

36. A method of treating a fungal infection in a patient in need comprising administering to said patient an effective amount of a composition according to claim 30.

37. The method of claim 35 wherein said compound is co-administered with at least one additional antifungal agent.

38. The method of claim 37 wherein said additional antifungal agent is selected from the group consisting of a polyene, imidazole, triazole, allylamine, echinocandin, a miscellaneous antifungal agent or a mixture thereof

39. The method according to claim 37 wherein said wherein said antifungal agent is selected from the group consisting of nystatin, amphotericin B, ketoconazole, clotrimazole, fluconazole, itraconazole, posaconazole, voriconazole, terbinafine, anidulafungin, caspofungin, micafungin, flucytosine, griseofulvin, pentamine or a mixture thereof.

40. The method according to claim 35 wherein said fungal infection is a dermatological fungal disease and/or condition, a respiratory fungal disease and/or condition, a neurological fungal disease and/or condition or a hepatic fungal disease and/or condition.

41. The method according to claim 35 wherein said fungal disease and/or condition is tinea versicolor, athlete's foot (Tinea pedis), jock itch (Tinea cruris), ringworm of the body (Tinea corporis), tinea of the beard (Tinea barbae), Tinea of the scalp (Tinea capitis), Histoplasmosis, Blastomycosis, Coccidiodomycosis, Paracoccidiodomycosis, Cryptococcosis, Aspergillosis, Zygomycosis, Candidiasis, Pneumocystis pneumonia, meningitis, Brain Abscess, Histoplasmosis or Candidiasis of the kidneys.

42. Use of a compound according to claim 1 in the manufacture of a medicament for use in the treatment of a fungal infection and/or condition.

43. Use according to claim 42 further including an additional antifungal agent.

44. Use of a composition according to claim 30 in the manufacture of a medicament for the treatment of a fungal infection and/or condition.
